SEQUENCE LISTING (20 sequences)

SEQ ID NO: 1 - DNA, 7596 bp, Mus musculus
  CDS (1)..(7596)
atgccacggctcctgacgcccttgctctgcctaacgctgctgcccgcgctcgccgcaagaggcttgagatgctcccagccaagtgggacctgcctgaatggaggtaggtgcgaagtggccagcggcactgaagcctgtgtctgcagcggagcctttgtgggccaacgatgccaggactccaatccttgcctcagcacaccgtgtaagaatgctggaacgtgccacgttgtggaccatggtggcactgtggattatgcctgcagctgtcccctgggtttctctgggcccctctgcctgacacctctggacaacgcctgcctggccaacccctgccgcaatgggggcacctgtgacctgctcactctcacagagtacaagtgccgctgcccaccagggtggtcaggaaaatcatgtcagcaggctgacccctgtgcctccaacccctgtgccaatggtggccagtgcctgccctttgagtcttcatacatctgtcgctgcccgcctggcttccatggccccacctgcaggcaagatgttaatgagtgcagccagaaccctgggctgtgccgccatggaggcacctgccacaatgagatcggctcctatcgctgtgcctgccgtgccacccatactggtccccactgtgaactgccctatgtgccctgcagcccctcaccctgccagaatggaggcacctgccgtcctacaggggacaccacccacgagtgtgcctgcttgccaggttttgctggacagaactgtgaagaaaatgtggatgactgtccaggaaacaactgcaagaatgggggtgcctgtgtggacggcgtgaatacctacaattgccgctgcccaccggagtggacgggtcagtactgtacagaggatgtggacgaatgtcagctcatgcccaatgcctgccagaatggcggaacctgccacaacacacacggcggctacaactgtgtgtgtgtcaatgggtggactggcgaggactgcagtgagaacattgatgactgtgccagtgccgcctgtttccagggtgccacttgccacgaccgtgtggcttccttctactgcgaatgtccgcatgggcgcacaggtctgctgtgccacctcaacgatgcgtgcatcagcaacccctgcaacgagggctccaactgtgacaccaaccctgtcaacggcaaagccatctgcacctgcccctcggggtacacagggccagcctgcagccaggacgtggatgagtgtgctctgggtgccaacccttgtgagcacgcaggcaaatgcctcaacacactgggttcttttgagtgccagtgtctacagggctacacgggaccccgctgtgagattgatgttaatgagtgcatctccaacccatgtcagaatgatgccacttgcctggaccagattggggagttccaatgcatatgtatgccaggttatgaaggtgtatactgtgaaatcaacacggatgagtgcgccagcagcccctgtctgcacaatggccactgcatggacaagatcaatgagttccaatgtcagtgccccaaaggcttcaacgggcacctgtgccagtatgatgtggatgagtgtgccagcacaccatgcaagaacggtgccaagtgcctggatgggcccaacacctatacctgcgtgtgtacagaaggttacacagggacccactgcgaagtggacattgacgagtgtgaccctgacccctgccactatggttcctgtaaggatggtgtggccacctttacctgcctgtgccagccaggctacacaggccatcactgtgagaccaacatcaatgagtgccacagccaaccgtgccgccatgggggcacctgccaggaccgtgacaactcctacctctgcttatgcctcaagggaaccacagggcccaactgtgagatcaacctggatgactgcgccagcaacccctgtgactctggcacctgtctggacaagattgatggctacgaatgtgcctgtgaaccaggctacacaggaagcatgtgtaacgtcaacattgacgaatgtgcgggcagcccctgccacaacgggggcacttgtgaggatggcatcgcgggcttcacttgccgctgccccgagggctaccatgaccccacgtgcctgtccgaggtcaacgagtgcaacagtaacccctgcatccacggagcttgccgggatggcctcaatgggtacaagtgtgactgtgcccctgggtggagtggaacaaactgtgacatcaacaacaacgagtgtgagtccaacccttgtgtcaacggtggcacctgcaaggacatgaccagtggctacgtatgcacctgccgagaaggcttcagtggccctaattgccagaccaacatcaacgaatgtgcctccaacccctgcctgaaccaggggacctgcattgatgatgtcgctggatacaagtgcaactgtcctctgccatatacaggagccacgtgtgaggtggtgttggccccatgtgctaccagcccctgcaaaaacagcggggtatgcaaggagtctgaagactatgagagtttttcctgtgtctgtcccacaggctggcaaggtcaaacctgcgaggttgacatcaatgagtgtgtgaaaagcccatgtcgccatggggcctcctgccagaacaccaatggcagctaccgctgcctctgccaggccggctatacaggtcgcaactgtgagagtgacatcgatgactgccgccccaacccgtgtcacaatgggggttcctgcaccgatggcatcaacacagccttctgcgactgcctgcccggcttccagggtgccttctgtgaggaggacatcaatgaatgtgccagcaatccctgccaaaatggtgccaattgcactgactgtgtggacagctacacatgtacctgccccgtgggcttcaatggcatccactgcgagaacaacacacctgactgtactgagagctcctgcttcaatggtggtacctgtgtggatggtatcaactccttcacctgtctgtgtccacctggcttcacgggcagctactgtcagtatgatgtcaatgagtgtgattcacggccctgtctgcacggtggtacctgccaagacagctatggtacttataagtgtacctgcccacagggctacactggtctcaactgccagaaccttgtgcgctggtgcgactcggctccctgcaagaatggtggcaggtgctggcagaccaacacgcagtaccactgtgagtgccgcagcggctggactggcgtcaactgcgacgtgctcagtgtgtcctgtgaggtggctgcacagaagcgaggcattgacgtcactctcctgtgccagcatggagggctctgtgtggatgagggagataaacattactgccactgccaggcaggctacacgggcagctactgtgaggacgaggtggacgagtgctcacctaacccctgccagaatggagctacctgcactgactatctcggcggcttttcctgcaagtgtgtggctggctaccatgggtctaactgctcagaggagatcaacgagtgcctgtcccagccctgccagaatgggggtacctgcattgatctgaccaactcctacaagtgttcctgcccccgggggacacagggtgtacactgtgagatcaatgttgatgactgccatcccccccttgaccctgcctcccgaagccccaagtgcttcaacaatggcacctgtgtggaccaggtgggtggctatacctgcacctgcccaccaggcttcgtcggggagcggtgtgagggtgatgtcaatgaatgtctctccaacccctgtgacccacgtggcacccagaactgtgtgcagcgtgttaatgacttccactgcgagtgccgggctggccacactggacgccgctgtgagtcagtcatcaatggctgcaggggcaaaccttgcaagaatgggggtgtctgtgccgtggcctccaacaccgcccgtggattcatctgtaggtgccctgcgggcttcgagggtgccacatgtgagaatgatgcccgcacttgtggcagcttacgctgcctcaacggtggtacatgcatctcgggcccacgtagtcccacctgcctatgcctgggatccttcaccggccctgagtgccagttcccagccagcagcccctgtgtgggtagcaacccctgctacaatcagggcacctgtgagcccacatccgagaaccctttctaccgctgtctatgccctgccaaattcaacgggctactgtgccacatcctggactacagcttcacaggtggcgctgggcgcgacattcccccaccgcagattgaggaggcctgtgagctgcctgagtgccaggtggatgcaggcaataaggtctgcaacctgcagtgtaataatcacgcatgtggctgggatggtggcgactgctccctcaacttcaatgacccctggaagaactgcacgcagtctctacagtgctggaagtattttagcgacggccactgtgacagccagtgcaactcggccggctgcctctttgatggcttcgactgccagctcaccgagggacagtgcaaccccctgtatgaccagtactgcaaggaccacttcagtgatggccactgcgaccagggctgtaacagtgccgaatgtgagtgggatggcctagactgtgctgagcatgtacccgagcggctggcagccggcaccctggtgctggtggtgctgcttccacccgaccagctacggaacaactccttccactttctgcgggagctcagccacgtgctgcacaccaacgtggtcttcaagcgtgatgcgcaaggccagcagatgatcttcccgtactatggccacgaggaagagctgcgcaagcacccaatcaagcgctctacagtgggttgggccacctcttcactgcttcctggtaccagtggtgggcgccagcgcagggagctggaccccatggacatccgtggctccattgtctacctggagatcgacaaccggcaatgtgtgcagtcatcctcgcagtgcttccagagtgccaccgatgtggctgccttcctaggtgctcttgcgtcacttggcagcctcaatattccttacaagattgaggccgtgaagagtgagccggtggagcctccgctgccctcgcagctgcacctcatgtacgtggcagcggccgccttcgtgctcctgttctttgtgggctgtggggtgctgctgtcccgcaagcgccggcggcagcatggccagctctggttccctgagggtttcaaagtgtcagaggccagcaagaagaagcggagagagcccctcggcgaggactcagtcggcctcaagcccctgaagaatgcctcagatggtgctctgatggacgacaatcagaacgagtggggagacgaagacctggagaccaagaagttccggtttgaggagccagtagttctccctgacctgagtgatcagactgaccacagacagtggacccagcagcacctggacgctgctgacctgcgcatgtctgccatggccccaacaccgcctcagggggaggtggatgctgactgcatggatgtcaatgttcgaggaccagatggcttcacacccctcatgattgcctcctgcagtggagggggccttgagacaggcaacagtgaagaagaagaagatgcacctgctgtcatctctgacttcatctaccagggcgccagcttgcacaaccagacagaccgcaccggggagaccgccttgcacttggctgcccgatactctcgttcagatgctgcaaagcgcttgctggaggccagtgcagatgccaacatccaggacaacatgggccgtactccgttacatgcagcagtttctgcagatgctcagggtgtcttccagatcctgctccggaacagggccacagatctggatgcccgaatgcatgatggcacaactccactgatcctggctgcgcgcctggccctggagggcatgctggaggacctcatcaactcacatgctgacgtcaatgccgtggatgacctaggcaagtcggctttgcattgggcggccgcggtgaacaatgtggatgctgctgttgtgctcctgaagaacggagccaacaaggacatgcagaacaacaaggaggagactcccctgttcctggccgcccgtgagggcagctatgagactgccaaagtgttgctggaccactttgccaaccgggacatcacggatcacatggaccgattgccgcgggacatcgcacaggagcgtatgcaccacgatatcgtgcggcttttggatgagtacaacctggtgcgcagcccacagctgcatggcactgccctgggtggcacacccactctgtctcccacactctgctcgcccaatggctacctgggcaatctcaagtctgccacacagggcaagaaggcccgcaagcccagcaccaaagggctggcttgtggtagcaaggaagctaaggacctcaaggcacggaggaagaagtcccaggatggcaagggctgcctgttggacagctcgagcatgctgtcgcctgtggactccctcgagtcaccccatggctacttgtcagatgtggcctcgccacccctcctcccctccccattccagcagtctccatccatgcctctcagccacctgcctggtatgcctgacactcacctgggcatcagccacttgaatgtggcagccaagcctgagatggcagcactggctggaggtagccggttggcctttgagccacccccgccacgcctctcccacctgcctgtagcctccagtgccagcacagtgctgagtaccaatggcacgggggctatgaatttcaccgtgggtgcaccggcaagcttgaatggccagtgtgagtggcttccccggctccagaatggcatggtgcccagccagtacaacccactacggccgggtgtgacgccgggcacactgagcacacaggcagctggcctccagcatagcatgatggggccactacacagcagcctctccaccaataccttgtccccgattatttaccagggcctgcccaacacacggctggcaacacagcctcacctggtgcagacccagcaggtgcagccacagaacttacagctccagcctcagaacctgcagccaccatcacagccacacctcagtgtgagctcggcagccaatgggcacctgggccggagcttcttgagtggggagcccagtcaggcagatgtacaaccgctgggccccagcagtctgcctgtgcacaccattctgccccaggaaagccaggccctgcccacatcactgccatcctccatggtcccacccatgaccactacccagttcctgacccctccttcccagcacagttactcctcctcccctgtggacaacacccccagccaccagctgcaggtgccagagcaccccttcctcaccccatcccctgagtcccctgaccagtggtccagctcctccccgcattccaacatctctgattggtccgagggcatctccagcccgcccaccaccatgccgtcccagatcacccacattccagaggcatttaaataa

SEQ ID NO: 2 - protein, 2531 aa, Mus musculus
MPRLLTPLLCLTLLPALAARGLRCSQPSGTCLNGGRCEVASGTEACVCSGAFVGQRCQDSNPCLSTPCKNAGTCHVVDHGGTVDYACSCPLGFSGPLCLTPLDNACLANPCRNGGTCDLLTLTEYKCRCPPGWSGKSCQQADPCASNPCANGGQCLPFESSYICRCPPGFHGPTCRQDVNECSQNPGLCRHGGTCHNEIGSYRCACRATHTGPHCELPYVPCSPSPCQNGGTCRPTGDTTHECACLPGFAGQNCEENVDDCPGNNCKNGGACVDGVNTYNCRCPPEWTGQYCTEDVDECQLMPNACQNGGTCHNTHGGYNCVCVNGWTGEDCSENIDDCASAACFQGATCHDRVASFYCECPHGRTGLLCHLNDACISNPCNEGSNCDTNPVNGKAICTCPSGYTGPACSQDVDECALGANPCEHAGKCLNTLGSFECQCLQGYTGPRCEIDVNECISNPCQNDATCLDQIGEFQCICMPGYEGVYCEINTDECASSPCLHNGHCMDKINEFQCQCPKGFNGHLCQYDVDECASTPCKNGAKCLDGPNTYTCVCTEGYTGTHCEVDIDECDPDPCHYGSCKDGVATFTCLCQPGYTGHHCETNINECHSQPCRHGGTCQDRDNSYLCLCLKGTTGPNCEINLDDCASNPCDSGTCLDKIDGYECACEPGYTGSMCNVNIDECAGSPCHNGGTCEDGIAGFTCRCPEGYHDPTCLSEVNECNSNPCIHGACRDGLNGYKCDCAPGWSGTNCDINNNECESNPCVNGGTCKDMTSGYVCTCREGFSGPNCQTNINECASNPCLNQGTCIDDVAGYKCNCPLPYTGATCEVVLAPCATSPCKNSGVCKESEDYESFSCVCPTGWQGQTCEVDINECVKSPCRHGASCQNTNGSYRCLCQAGYTGRNCESDIDDCRPNPCHNGGSCTDGINTAFCDCLPGFQGAFCEEDINECASNPCQNGANCTDCVDSYTCTCPVGFNGIHCENNTPDCTESSCFNGGTCVDGINSFTCLCPPGFTGSYCQYDVNECDSRPCLHGGTCQDSYGTYKCTCPQGYTGLNCQNLVRWCDSAPCKNGGRCWQTNTQYHCECRSGWTGVNCDVLSVSCEVAAQKRGIDVTLLCQHGGLCVDEGDKHYCHCQAGYTGSYCEDEVDECSPNPCQNGATCTDYLGGFSCKCVAGYHGSNCSEEINECLSQPCQNGGTCIDLTNSYKCSCPRGTQGVHCEINVDDCHPPLDPASRSPKCFNNGTCVDQVGGYTCTCPPGFVGERCEGDVNECLSNPCDPRGTQNCVQRVNDFHCECRAGHTGRRCESVINGCRGKPCKNGGVCAVASNTARGFICRCPAGFEGATCENDARTCGSLRCLNGGTCISGPRSPTCLCLGSFTGPECQFPASSPCVGSNPCYNQGTCEPTSENPFYRCLCPAKFNGLLCHILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLSDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILLRNRATDLDARMHDGTTPLILAARLALEGMLEDLINSHADVNAVDDLGKSALHWAAAVNNVDAAVVLLKNGANKDMQNNKEETPLFLAAREGSYETAKVLLDHFANRDITDHMDRLPRDIAQERMHHDIVRLLDEYNLVRSPQLHGTALGGTPTLSPTLCSPNGYLGNLKSATQGKKARKPSTKGLACGSKEAKDLKARRKKSQDGKGCLLDSSSMLSPVDSLESPHGYLSDVASPPLLPSPFQQSPSMPLSHLPGMPDTHLGISHLNVAAKPEMAALAGGSRLAFEPPPPRLSHLPVASSASTVLSTNGTGAMNFTVGAPASLNGQCEWLPRLQNGMVPSQYNPLRPGVTPGTLSTQAAGLQHSMMGPLHSSLSTNTLSPIIYQGLPNTRLATQPHLVQTQQVQPQNLQLQPQNLQPPSQPHLSVSSAANGHLGRSFLSGEPSQADVQPLGPSSLPVHTILPQESQALPTSLPSSMVPPMTTTQFLTPPSQHSYSSSPVDNTPSHQLQVPEHPFLTPSPESPDQWSSSSPHSNISDWSEGISSPPTTMPSQITHIPEAFK

SEQ ID NO: 3 - DNA, 2346 bp, Mus musculus
  CDS (1)..(2346)
cggcggcagcatggccagctctggttccctgagggtttcaaagtgtcagaggccagcaagaagaagcggagagagcccctcggcgaggactcagtcggcctcaagcccctgaagaatgcctcagatggtgctctgatggacgacaatcagaacgagtggggagacgaagacctggagaccaagaagttccggtttgaggagccagtagttctccctgacctgagtgatcagactgaccacagacagtggacccagcagcacctggacgctgctgacctgcgcatgtctgccatggccccaacaccgcctcagggggaggtggatgctgactgcatggatgtcaatgttcgaggaccagatggcttcacacccctcatgattgcctcctgcagtggagggggccttgagacaggcaacagtgaagaagaagaagatgcacctgctgtcatctctgacttcatctaccagggcgccagcttgcacaaccagacagaccgcaccggggagaccgccttgcacttggctgcccgatactctcgttcagatgctgcaaagcgcttgctggaggccagtgcagatgccaacatccaggacaacatgggccgtactccgttacatgcagcagtttctgcagatgctcagggtgtcttccagatcctgctccggaacagggccacagatctggatgcccgaatgcatgatggcacaactccactgatcctggctgcgcgcctggccctggagggcatgctggaggacctcatcaactcacatgctgacgtcaatgccgtggatgacctaggcaagtcggctttgcattgggcggccgcggtgaacaatgtggatgctgctgttgtgctcctgaagaacggagccaacaaggacatgcagaacaacaaggaggagactcccctgttcctggccgcccgtgagggcagctatgagactgccaaagtgttgctggaccactttgccaaccgggacatcacggatcacatggaccgattgccgcgggacatcgcacaggagcgtatgcaccacgatatcgtgcggcttttggatgagtacaacctggtgcgcagcccacagctgcatggcactgccctgggtggcacacccactctgtctcccacactctgctcgcccaatggctacctgggcaatctcaagtctgccacacagggcaagaaggcccgcaagcccagcaccaaagggctggcttgtggtagcaaggaagctaaggacctcaaggcacggaggaagaagtcccaggatggcaagggctgcctgttggacagctcgagcatgctgtcgcctgtggactccctcgagtcaccccatggctacttgtcagatgtggcctcgccacccctcctcccctccccattccagcagtctccatccatgcctctcagccacctgcctggtatgcctgacactcacctgggcatcagccacttgaatgtggcagccaagcctgagatggcagcactggctggaggtagccggttggcctttgagccacccccgccacgcctctcccacctgcctgtagcctccagtgccagcacagtgctgagtaccaatggcacgggggctatgaatttcaccgtgggtgcaccggcaagcttgaatggccagtgtgagtggcttccccggctccagaatggcatggtgcccagccagtacaacccactacggccgggtgtgacgccgggcacactgagcacacaggcagctggcctccagcatagcatgatggggccactacacagcagcctctccaccaataccttgtccccgattatttaccagggcctgcccaacacacggctggcaacacagcctcacctggtgcagacccagcaggtgcagccacagaacttacagctccagcctcagaacctgcagccaccatcacagccacacctcagtgtgagctcggcagccaatgggcacctgggccggagcttcttgagtggggagcccagtcaggcagatgtacaaccgctgggccccagcagtctgcctgtgcacaccattctgccccaggaaagccaggccctgcccacatcactgccatcctccatggtcccacccatgaccactacccagttcctgacccctccttcccagcacagttactcctcctcccctgtggacaacacccccagccaccagctgcaggtgccagagcaccccttcctcaccccatcccctgagtcccctgaccagtggtccagctcctccccgcattccaacatctctgattggtccgagggcatctccagcccgcccaccaccatgccgtcccagatcacccacattccagaggcatttaaataa

SEQ ID NO: 4 - protein, 781 aa, Mus musculus
RRQHGQLWFPEGFKVSEASKKKRREPLGEDSVGLKPLKNASDGALMDDNQNEWGDEDLETKKFRFEEPVVLPDLSDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILLRNRATDLDARMHDGTTPLILAARLALEGMLEDLINSHADVNAVDDLGKSALHWAAAVNNVDAAVVLLKNGANKDMQNNKEETPLFLAAREGSYETAKVLLDHFANRDITDHMDRLPRDIAQERMHHDIVRLLDEYNLVRSPQLHGTALGGTPTLSPTLCSPNGYLGNLKSATQGKKARKPSTKGLACGSKEAKDLKARRKKSQDGKGCLLDSSSMLSPVDSLESPHGYLSDVASPPLLPSPFQQSPSMPLSHLPGMPDTHLGISHLNVAAKPEMAALAGGSRLAFEPPPPRLSHLPVASSASTVLSTNGTGAMNFTVGAPASLNGQCEWLPRLQNGMVPSQYNPLRPGVTPGTLSTQAAGLQHSMMGPLHSSLSTNTLSPIIYQGLPNTRLATQPHLVQTQQVQPQNLQLQPQNLQPPSQPHLSVSSAANGHLGRSFLSGEPSQADVQPLGPSSLPVHTILPQESQALPTSLPSSMVPPMTTTQFLTPPSQHSYSSSPVDNTPSHQLQVPEHPFLTPSPESPDQWSSSSPHSNISDWSEGISSPPTTMPSQITHIPEAFK

SEQ ID NO: 5 - DNA, 900 bp, Mus musculus
  CDS (1)..(900)
atgaagagagctcacccagactacagctcctcagatagtgagctggacgagaccatcgaggtggaaaaggagagcgcggacgagaatggaaacttgagttcggcgctgtgttccatgtccccaacgacatcgtcccaggttttggccaggaaaagacggagaggcatcatcgagaagcgccgacgagaccgaatcaataacagtttgtctgagctgagaaggctggtacccagtgcctttgagaagcagggatctgctaagctagaaaaagctgagatcttgcagatgactgtggatcacctgaaaatgctgcacactgcaggagggaaaggttattttgacgcgcacgccctggctatggactatcggagtttggggtttcgggaatgcctggccgaagttgcccgttatctgagcatcattgaaggactcgatgcctccgacccgcttcgcgttcgcctggtctcccatctcaacaactacgcatcccagcgggaagccgcgagcggcgctcacggtggcctcggacacattccctggggaagtgccttcggacatcacccacacatcgcacaccctctgctgctgccccagaatggccacgggaacgctggcaccgcggcgtcacccacggagccgcatcaccagggcaggctggcttctgcccatccggaggcgccggccttgcgagcgccccctagtggcggcttgggaccggtgcttcccgtggtgacctcggcctccaaactgtctccaccgctgctctcctctgtggcctcgctctcagccttccccttttccttcagctccttccacctactgagcccttcgacacccacgcaggcagcaaaccttggcaagccctatagaccttgggggacagagatcggagctttctaa

SEQ ID NO: 6 - protein, 299 aa, Mus musculus
MKRAHPDYSSSDSELDETIEVEKESADENGNLSSALCSMSPTTSSQVLARKRRRGIIEKRRRDRINNSLSELRRLVPSAFEKQGSAKLEKAEILQMTVDHLKMLHTAGGKGYFDAHALAMDYRSLGFRECLAEVARYLSIIEGLDASDPLRVRLVSHLNNYASQREAASGAHGGLGHIPWGSAFGHHPHIAHPLLLPQNGHGNAGTAASPTEPHHQGRLASAHPEAPALRAPPSGGLGPVLPVVTSASKLSPPLLSSVASLSAFPFSFSSFHLLSPSTPTQAANLGKPYRPWGTEIGAF

SEQ ID NO: 7 - DNA, 1020 bp, Mus musculus
  CDS (1)..(1020)
atgaagcgcccttgtgaggaaacgacctccgaaagcgacctggacgagaccatcgacgtggggagcgagaacaattaccctgggcacgctacaagctcagtgatgaggtccaattcaccgacaactacctctcagattatggcaagaaagaaaaggagagggatcatagaaaaaaggcgtcgggatcgaataaataacagtttatctgaattgagaagactagtgccaacagcttttgaaaaacaaggatctgccaagttagaaaaggctgaaatattgcaaatgacagtggatcatttgaagatgctccaggctacagggggtaaaggctactttgatgcccatgctcttgccacagacttcatgagcattggattccgagagtgcttgacagaagtggctaggtacctaagctcagtggaaggccttgacccgtcggacccactacgcgtgcgccttgtctctcatctcagcacctgtgcctcccagcgggaggcagcagtgatgacatcctccatggcccaccaccatcaccccttgcaccctcaccactgggcagctgctttccaccatctccccacagccctgctccagcccaatggactccacacatcagagtcaaccccatgtcgcctatccacatcttcagaagtgccttctgctcatggctctgctctcctcacagcaacgtttgcccatgcagattctgctcttcggatgccatcagggggcaccgttgcaccctgcgtgccacctctctccacctctcttctgtctctttcggccactgtgcatgccgcagctgcagcagccactgcagctgcacacagcttccctctgtccttcgcaggggcttttcccatgctcccgtccaatgcagcggcagcagccgctgttgctgctgcaacagcaatcagcccacccttgtcggtatccgcagcctccagtcctcagcagacaagcactgggacaaacaataaaccttaccaaccctgggggacagaagttggagccttttaa

SEQ ID NO: 8 - protein, 339 aa, Mus musculus
MKRPCEETTSESDLDETIDVGSENNYPGHATSSVMRSNSPTTTSQIMARKKRRGIIEKRRRDRINNSLSELRRLVPTAFEKQGSAKLEKAEILQMTVDHLKMLQATGGKGYFDAHALATDFMSIGFRECLTEVARYLSSVEGLDPSDPLRVRLVSHLSTCASQREAAVMTSSMAHHHHPLHPHHWAAAFHHLPTALLQPNGLHTSESTPCRLSTSSEVPSAHGSALLTATFAHADSALRMPSGGTVAPCVPPLSTSLLSLSATVHAAAAAATAAAHSFPLSFAGAFPMLPSNAAAAAAVAAATAISPPLSVSAASSPQQTSTGTNNKPYQPWGTEVGAF

SEQ ID NO: 9 - DNA, 849 bp, Mus musculus
  CDS (1)..(849)
atgccagctgatataatggagaaaaattcctcctccccggtggctgctaccccagccagtgtcaacacgacaccggacaaaccaaagacggcctctgagcacagaaagtcatcaaagcctatcatggagaagaggcgaagggcaagaataaatgaaagtctaagccaactgaaaacactgattttggatgcacttaagaaagatagctcccggcattccaagctagagaaggcagacattctggaaatgactgtgaagcacctccggaacctgcagcgggcgcagatgaccgccgcgctcagcacagacccgagcgtgttggggaaataccgcgccggcttcagcgaagagtgcatgagcgaggtgacgcgtttcctgtccacgtgtgagggcgttaccaccgaggtgctcactcggctaatgggccacctggccagttgcatgacccagatcatcgccatgtaccccgggcaggcgcaccccgccttgcaggcgccgccgccgccgcccccgtcaggacctgccggtccccagcacgcgccattcgcgccgccgccgccgccgcttgtgcccatccccgggggcgcggcgccccctcccggcagcgcaccctgcaagttgggcagccaggctggagaggctgccaaggtttttggcggcttccaagtggtgccggctcctgacggccaatttgcctttctcatccccaacggggccttcgctcacagcggcccggtcatcccggtctacaccagcaacagtgggacctcggtgggtcctaacgcagtgtcaccttccagtggctcctcgctcacttcggactccatgtggagaccgtggcggaactga

SEQ ID NO: 10 - protein, 282 aa, Mus musculus
MPADIMEKNSSSPVAATPASVNTTPDKPKTASEHRKSSKPIMEKRRRARINESLSQLKTLILDALKKDSSRHSKLEKADILEMTVKHLRNLQRAQMTAALSTDPSVLGKYRAGFSEECMSEVTRFLSTCEGVTTEVLTRLMGHLASCMTQIIAMYPGQAHPALQAPPPPPPSGPAGPQHAPFAPPPPPLVPIPGGAAPPPGSAPCKLGSQAGEAAKVFGGFQVVPAPDGQFAFLIPNGAFAHSGPVIPVYTSNSGTSVGPNAVSPSSGSSLTSDSMWRPWRN

SEQ ID NO: 11 - DNA, 528 bp, Mus musculus
  CDS (1)..(528)
atggagaagaagcgccgtgcacgcatcaacgtgtcactggagcagctgaggtctcttctggagagacactactcacatcagatacggaaacgaaagctggagaaggccgatatcctggagctgagtgttaagtacatgagaagcctccagaactcactgcaaggactctggccagtacccagtggggtggactacccgtccggcttccaaggcggcttgcgtggcgtgagccagaggcttcggcccggagagggagacagcggcctgcgctgccccttgctgctccagcgcagggaaggcagcaccacggacagcgccaacccacaggcgacctctgttctcaacccttgcctcccggccatctgggcccccagtcgcgctgcaggtggctcccactccccgcagtccccgctccctctccctggaggtctccttgagtcttccaccgatgttgtggcaccacatcctgcatcaaactgccaggccgagagtacgagacccgggtttcgcgtgtggcgaccgtggtga

SEQ ID NO: 12 - protein, 175 aa, Mus musculus
MEKKRRARINVSLEQLRSLLERHYSHQIRKRKLEKADILELSVKYMRSLQNSLQGLWPVPSGVDYPSGFQGGLRGVSQRLRPGEGDSGLRCPLLLQRREGSTTDSANPQATSVLNPCLPAIWAPSRAAGGSHSPQSPLPLPGGLLESSTDVVAPHPASNCQAESTRPGFRVWRPW

SEQ ID NO: 13 - DNA, 504 bp, Mus musculus
  CDS (1)..(504)
atggccccaagtaccgtggcggtggagatgctcagtcccaaggagaaaaaccgactgcggaagccggtggtggagaagatgcgtcgggaccgcatcaacagcagcatagagcagctgaagctgctgctggagcaggagttcgcgcggcaccagcccaactccaagctggagaaggccgacatcctggagatggccgtcagctacctgaaacacagcaaagccttcgccgcggccgccggccccaagagcctgcaccaggactacagcgagggctactcctggtgcctgcaggaggcggtacagttcctgaccctgcacgccgccagcgacacgcagatgaagctgctttaccacttccagcggcccccagctcccgccgctcctgccaaggagcccccggcgcccggagctgcgccccaaccggcccgctcctccgctaaggctgctgctgctgcggtctccacctcgcgccaacccgcctgcggcctctggcggccctggtga

SEQ ID NO: 14 - protein, 167 aa, Mus musculus
MAPSTVAVEMLSPKEKNRLRKPVVEKMRRDRINSSIEQLKLLLEQEFARHQPNSKLEKADILEMAVSYLKHSKAFAAAAGPKSLHQDYSEGYSWCLQEAVQFLTLHAASDTQMKLLYHFQRPPAPAAPAKEPPAPGAAPQPARSSAKAAAAAVSTSRQPACGLWRPW

SEQ ID NO: 15 - DNA, 675 bp, Mus musculus
  CDS (1)..(675)
atggctccgtcccaggcgcccagccgggaccgtgcaggccaggaggatgaggaccgctgggaagcacggggggaccgcaaggcccggaagcccctggtggagaagaagcgacgcgcacggatcaacgagagtcttcaggagctgcggctgctgctggccggtaccgaggtgcaggccaagctagagaacgccgaggttctggagctgaccgtgaggcgcgtgcagggcgcgctgcgaggccgggcgcgcgagcgggagcagctgcaggctgaagcaagcgagcgcttcgctgctggctacatccagtgcatgcatgaggtgcacacgttcgtgtccacgtgccaagccatcgatgccactgtctcagctgaactcctgaaccacctgctagaatccatgccgctgcgtgagggtagcagctttcaggatctgctgggggactccctagctgggctgcctggaggctctgggaggagcagctggcctccaggagggtccccagaatccccattgtccagtcccccaggtcccggggacgacctgtgttctgacctagaggagatcccagaggctgaactaaaccgggtgcctgctgaggggccggatttggtgtctacatccttaggcagcctgactgcagctcgccgggctcagagtgtgtggaggccttggtga

SEQ ID NO: 16 - protein, 224 aa, Mus musculus
MAPSQAPSRDRAGQEDEDRWEARGDRKARKPLVEKKRRARINESLQELRLLLAGTEVQAKLENAEVLELTVRRVQGALRGRAREREQLQAEASERFAAGYIQCMHEVHTFVSTCQAIDATVSAELLNHLLESMPLREGSSFQDLLGDSLAGLPGGSGRSSWPPGGSPESPLSSPPGPGDDLCSDLEEIPEAELNRVPAEGPDLVSTSLGSLTAARRAQSVWRPW

SEQ ID NO: 17 - protein, 792 aa, Artificial sequence
  Synthetic
RRQHGQLWFPEGFKVSEASKKKRREPLGEDSVGLKPLKNASDGALMDDNQNEWGDEDLETKKFRFEEPVVLPDLSDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILLRNRATDLDARMHDGTTPLILAARLALEGMLEDLINSHADVNAVDDLGKSALHWAAAVNNVDAAVVLLKNGANKDMQNNKEETPLFLAAREGSYETAKVLLDHFANRDITDHMDRLPRDIAQERMHHDIVRLLDEYNLVRSPQLHGTALGGTPTLSPTLCSPNGYLGNLKSATQGKKARKPSTKGLACGSKEAKDLKARRKKSQDGKGCLLDSSSMLSPVDSLESPHGYLSDVASPPLLPSPFQQSPSMPLSHLPGMPDTHLGISHLNVAAKPEMAALAGGSRLAFEPPPPRLSHLPVASSASTVLSTNGTGAMNFTVGAPASLNGQCEWLPRLQNGMVPSQYNPLRPGVTPGTLSTQAAGLQHSMMGPLHSSLSTNTLSPIIYQGLPNTRLATQPHLVQTQQVQPQNLQLQPQNLQPPSQPHLSVSSAANGHLGRSFLSGEPSQADVQPLGPSSLPVHTILPQESQALPTSLPSSMVPPMTTTQFLTPPSQHSYSSSPVDNTPSHQLQVPEHPFLTPSPESPDQWSSSSPHSNISDWSEGISSPPTTMPSQITHIPEAFKYGRKKRRQRRR

SEQ ID NO: 18 - protein, 797 aa, Artificial sequence
  Synthetic
RRQHGQLWFPEGFKVSEASKKKRREPLGEDSVGLKPLKNASDGALMDDNQNEWGDEDLETKKFRFEEPVVLPDLSDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILLRNRATDLDARMHDGTTPLILAARLALEGMLEDLINSHADVNAVDDLGKSALHWAAAVNNVDAAVVLLKNGANKDMQNNKEETPLFLAAREGSYETAKVLLDHFANRDITDHMDRLPRDIAQERMHHDIVRLLDEYNLVRSPQLHGTALGGTPTLSPTLCSPNGYLGNLKSATQGKKARKPSTKGLACGSKEAKDLKARRKKSQDGKGCLLDSSSMLSPVDSLESPHGYLSDVASPPLLPSPFQQSPSMPLSHLPGMPDTHLGISHLNVAAKPEMAALAGGSRLAFEPPPPRLSHLPVASSASTVLSTNGTGAMNFTVGAPASLNGQCEWLPRLQNGMVPSQYNPLRPGVTPGTLSTQAAGLQHSMMGPLHSSLSTNTLSPIIYQGLPNTRLATQPHLVQTQQVQPQNLQLQPQNLQPPSQPHLSVSSAANGHLGRSFLSGEPSQADVQPLGPSSLPVHTILPQESQALPTSLPSSMVPPMTTTQFLTPPSQHSYSSSPVDNTPSHQLQVPEHPFLTPSPESPDQWSSSSPHSNISDWSEGISSPPTTMPSQITHIPEAFKRQIKIWFQNRRMKWKK

SEQ ID NO: 19 - protein, 350 aa, Artificial sequence
  Synthetic
YGRKKRRQRRRMKRPCEETTSESDLDETIDVGSENNYPGHATSSVMRSNSPTTTSQIMARKKRRGIIEKRRRDRINNSLSELRRLVPTAFEKQGSAKLEKAEILQMTVDHLKMLQATGGKGYFDAHALATDFMSIGFRECLTEVARYLSSVEGLDPSDPLRVRLVSHLSTCASQREAAVMTSSMAHHHHPLHPHHWAAAFHHLPTALLQPNGLHTSESTPCRLSTSSEVPSAHGSALLTATFAHADSALRMPSGGTVAPCVPPLSTSLLSLSATVHAAAAAATAAAHSFPLSFAGAFPMLPSNAAAAAAVAAATAISPPLSVSAASSPQQTSTGTNNKPYQPWGTEVGAF

SEQ ID NO: 20 - protein, 178 aa, Artificial Sequence
  Synthetic
YGRKKRRQRRRMAPSTVAVEMLSPKEKNRLRKPVVEKMRRDRINSSIEQLKLLLEQEFARHQPNSKLEKADILEMAVSYLKHSKAFAAAAGPKSLHQDYSEGYSWCLQEAVQFLTLHAASDTQMKLLYHFQRPPAPAAPAKEPPAPGAAPQPARSSAKAAAAAVSTSRQPACGLWRPW

(19) United States
(12) Patent Application Publication
(10) Pub. No.: US 2008/0248568 A1
(43) Pub. Date: Oct. 9, 2008

(54) Directed Neural Differentiation

(75) Inventors: Sarah Elizabeth Lowell, Edinburgh (GB); Austin Gerard Smith, Edinburgh (GB); Barry Heavey, Athlone (IE)

Correspondence Address: STERNE, KESSLER, GOLDSTEIN & FOX P.L.L.C., 1100 NEW YORK AVENUE, N.W., WASHINGTON, DC, 20005, US

(73) Assignee: The University Court of the University of Edinburgh

(21) Appl. No.: 11/908,276

(22) PCT Filed: Mar. 9, 2006
(86) PCT No.: PCT/GB06/00836

§ 371 Date: Sep. 10, 2007

(30) Foreign Application Priority Data
Mar. 9, 2005 (GB) 0504881.4

(51) Int. Cl. C12N 5/08; C12N 5/02; C07K 14/00; C07H 21/04; C12N 15/00

(52) U.S. Cl. 435/368; 435/375; 530/350; 536/23.4; 435/320.1; 435/404

(57) ABSTRACT

Differentiation towards a neural fate, and away from a non-neural fate, is promoted by activation of Notch signalling in ES cells and then transferring the cells into neural differentiation protocols. Media for neural differentiation comprises a Notch activator, e.g. a notch ligand that can be clustered. Genetic manipulation is used as an alternative to media additives for Notch activation.

The present invention relates to directed neural differentiation. In particular this invention relates to promoting differentiation of pluripotent cells towards a neural fate, reducing differentiation towards a non-neural fate and maintaining cultures of neural cells, and to methods and compositions therefor.

An important goal in stem cell research is to reliably convert embryonic stem (ES) cells into the neural lineage. This is important for two reasons: to allow us to better understand the mechanism of neural specification and to provide neural cells and tissue, e.g. for regenerative therapies, with minimal contamination by non-neural cells.

Recent progress towards this goal came with the identification of culture conditions under which a large proportion of ES cells become neural progenitors (Ying et al., 2003b). The two central features of these culture conditions are that they support survival of both ES and neural cells, and that they lack a source of exogenous BMP, which is a critical inhibitor of neural specification for ES cells (and which can be provided by serum)(Ying et al., 2003a).

However, even under these optimised culture conditions, between 20 and 40% of cells resist neural specification: around half of these differentiate into non-neural lineages, whilst the others remain as undifferentiated ES cells.

Notch signalling is known in many different tissues to regulate differentiation decisions by mediating signalling between neighbouring cells (Lai, 2004). Notch receptors and ligands are expressed in ES cells, but their function in this context is hitherto unknown. Notch ligands include members of two closely related families called Delta (Delta 1, 3 and 4) and Jagged (Jagged is also sometimes referred to as Serrate and 2 forms, Jagged1 and Jagged2, have been identified). They are all transmembrane proteins that sit on the cell surface and bind and activate Notch receptors on neighbouring cells.

It is known that the effect of Notch on lineage committed cells is generally to inhibit differentiation. Thus, ES cells have been generated with a targeted deletion of RBPJk, the co-activator that is required for Notch to activate transcription of its target genes and is thought to mediate the activity of all 4 Notch receptors (Schroeder et al., 2003). These ES cells have been successfully differentiated into the mesodermal lineage—the focus of this study was to uncover a bias in the subsequent differentiation of ES-derived mesoderm towards cardiomyocyte and away from other mesodermal lineages.

In another study, RBPJk-deficient ES cells were able to generate neurospheres (Hitoshi et al., 2002). Unfortunately, neurosphere cultures are highly selective, making it difficult to draw conclusions about any quantitative effects on the overall rate of neural specification

Notch has also been studied in tissues; e.g. it is known to try to activate Notch signalling in neural cells by adding a Notch ligand-Fc fusion to cell culture medium and then adding an anti-Fc antibody to cluster the Notch ligands. But these studies are not relevant to the effect of Notch signalling on pluripotent cells.

It is desired to obtain pure populations of differentiated cells from ES cells, e.g. pure neural cell lines, and protocols exist for generating neural stem cells (NSCs) from ES cells. Even so, several days culture are required. It is desired to reduce this.

Even when NSCs are obtained, the cultures still contain high numbers of contaminating non-neural cells. In addition, many NSCs differentiate, leading to a less and less homogenous culture. Further culture control, e.g. to inhibit further differentiation until such is desired, would be an advantage.

Another problem with existing methods for culture of neural cells is that the cells are sensitive to small changes in culture conditions. Cell survival is often adversely affected by variation in, e.g., plating density.

A further problem with trying to derive neural progeny from ES cells, particularly human ES cells, is that culture at high density tends to suppress neural induction, so any neural induction can only be done at low cell density and as a result low yield, even though lower cell density can lead to reduced cell survival.

An object of the invention is to improve the bias of differentiating pluripotent cell, especially ES cell, cultures towards a neural fate, and preferably achieve conversion at a rate of as close to 100% as possible.

A further object of specific embodiments of the invention is to promote differentiation towards a neural stem cell fate without pushing the cells all the way to terminal differentiation, e.g. it is desired to maintain cells at the neural stem cell stage after initial differentiation.

Another object of specific embodiments of the invention is to allow conversion of ES cells to neural cells, both of mouse and human as well as other origin, at higher densities.

A still further object of specific embodiments of the invention is to make the neural cell cultures more robust, increasing the survival of cells and reducing the effects of changes in culture conditions.

In the present invention, neural specification is promoted through activation of Notch signalling. Increased Notch signalling can hence be used to convert pluripotent cell cultures into cultures of neural cells, for example after withdrawal of BMP and/or serum during culture of ES or other pluripotent cells.

A first aspect of the invention provides use of Notch activation in promoting differentiation of multipotent or pluripotent cells towards a neural fate. The cells are preferably pluripotent stem cells, especially embryonic stem (ES) cells.

We have found that ES cells can be biased by Notch signalling to differentiate with reduced production of non-neural cells. This yields a purer neural cell culture with reduced contaminating cells.

To activate Notch signalling, one option is, constitutively or reversibly, to express an activated form of Notch, for example the Notch intracellular domain. In examples described below we have missexpressed Notch, in an activated form, and found this promotes neural specification in culture, even at high cell densities.

A second aspect of the invention provides use of Notch activation in suppressing differentiation of neural progenitor cells or neural stem cells into terminally differentiated neural cells, i.e. neurons and glia. We have used activation of Notch in neural cell cultures to prevent or at least delay differentiation of neural progenitors into terminally differentiated cells. This maintains the neural cell culture at the progenitor stage, enabling further purification to be undertaken before, e.g., actively promoting differentiation into the desired terminally differentiated cells. Control of the culture and of its purity is as a result improved.

The invention is of application generally to animal cells, including rodent and primate cells. In particular embodiments the invention is carried out to culture mouse or human neural cells

The invention also provides methods of culturing cells to obtain cultures containing neural cells or neural progenitor cells, and preferably free or substantially free of non-neural cells.

One such method of obtaining a culture enriched in neural progenitor cells, comprises:
(a) providing a culture comprising multipotent or pluripotent cells which have the potential to form progeny committed to either a neural or a non-neural fate; and
(b) activating Notch signalling in the cells.

The step of activating Notch is found to lead to increased neural specification in the culture, either as the sole means of directing differentiation towards a neural fate or in combination with other means, such as addition of factors known to stimulate neural specification.

Reference to Notch is reference to a transmembrane protein of that name, currently known to be family of 4 members, which acts as a receptor for various identified ligands and which has been reported to mediate cell fate selection via lateral inhibition. The terms “Notch” and “Notch receptor” may, dependent upon context, have the same meaning. Binding of ligand leads to cleavage of the protein to yield an intracellular, activated from of Notch referred to variously as an activated form of Notch, a Notch intracellular domain and an activated form of the Notch receptor. The intracellular domain complexes with RBPjk (also known as CBP) and the complex binds DNA in the nucleus and activates transcription of Notch target genes. Reference to Notch signalling and activation of Notch and activation of Notch signalling refers to activation of one or more signalling pathways that are mediated in vivo by an activated form of Notch and thus refers to increased transcription of target genes and/or other downstream effectors.

Notch signalling can thus be activated by expressing, in multipotent or pluripotent cells, an activated form of a Notch receptor, such as an intracellular domain of a Notch receptor. In examples we have performed, this activating has been genetically obtained, using a vector encoding an activated form of the receptor.

Notch signalling can also be activated by adding a Notch ligand, i.e. a ligand that binds to cell surface Notch receptor, to the culture medium. This approach may be preferred to others as it avoids genetic manipulation of the cells and can easily and quickly be adjusted and controlled.

A preferred Notch ligand, used in examples below and described in more detail therein, further comprises a tag, either intrinsic to the ligand or added e.g. in a fusion protein, and being a tag which can be bound by a further medium component, namely a clustering molecule. The clustering molecule binds to two or more tags and hence holds two or more ligands in close proximity to each other. The tag can also be referred to as a clustering epitope, such as a peptide sequence that an antibody will bind to.

A preferred method of the invention thus further comprises adding tagged ligand and clustering molecule to the culture medium. In use, the clustering molecule binds to two or more tags and holds the ligands together. The ligands bind to Notch receptors on the surface of cells in the culture, and the Notch receptors are as a result held closely together or clustered on the cell surface.

This clustering is part of activation of Notch signalling, and it is believed that Notch ligands may need to be attached to the cell membrane in order to successfully activate Notch—some attempts using specially engineered soluble forms of Notch ligands either have no effect or they inhibit rather than activate Notch signalling. The ligands may only be active when they are clustered together, and that clustering is facilitated by their localisation in a cell membrane. To achieve Notch activation using soluble ligands that do not cross the cell plasma membrane, a soluble ligand can therefore be combined with a tag that is bound by a separate clustering molecule that binds to tags on two or more ligands, hence forming a clustering molecule(Notch ligand)₂ complex and clustering the Notch ligands. For example, fusion proteins of Notch ligands with Fc sequences have been found effective as tagged ligands. Specifically, Carol Hicks in the laboratory of Gerry Weinmaster at UCLA has engineered fusion proteins with Delta or Jagged fused to an Fc molecule. Clustering is initiated by adding an anti-Fc antibody. The Delta-Fc or Jagged-Fc or other tagged ligand is added to the culture medium together with the Fc antibody. Another option is to provide the ligand in multivalent form—hence one ligand binds two or more receptors and clusters the receptors. Notch signaling was activated using a soluble ligand fused to a clustering molecule in Morrison et al., Cell, vol. 101, 26 May 2000, pp 499-510. Notch signaling was activated using F3 as ligand in Hu et al., Cell, vol. 115, 17 Oct. 2004, pp 163-175. Other known Notch ligands are Contactin, Lag2 and Serrate (another term for Jagged).

The soluble clustered Fc ligands can have different effects if too great an amount of ligand and/or antibody is used—as an excess can lead to antagonism of Notch signalling. The effect of the Fc-ligands can depend in particular upon the concentration of anti-Fc antibody used: too much antibody (thought to result in extra-large clusters) has the same effect as no antibody (no clustering) generating an antagonistic rather than agonistic effect. See: Hicks C, Ladi E, Lindsell C, Hsieh J J, Hayward S D, Collazo A, Weinmaster G. “A secreted Delta1-Fc fusion protein functions both as an activator and inhibitor of Notch1 signalling.” J Neurosci Res. 2002 Jun 15;68(6):655-67. As this is well known and documented it is straightforward for the skilled person to avoid excess amounts of the clustering molecule to ensure that only Notch activation is obtained.

Notch signaling can also be activated by expressing one or more downstream effectors of Notch signalling. For example a product of a gene directly or indirectly targeted by the Notch intracellular domain can be misexpressed. Thus, as an example, Notch signalling can be achieved by expression of a Hes or Hey gene, leading to increased activity of the Hes or Hey transcription factors. Notch activation can be achieved by increased activity of downstream targets of Hes and Hey genes. Notch activation was carried out by expressing Hey genes by Sakamoto et al. as described in J. Biological Chemistry, vol. 278, no. 45, issue of 7 Nov. 2004, pp 44808-44815. Generally, this expression can be achieved using a range of different vectors, such as a transfection vector or with a viral vector, e.g. lentivirus, retrovirus or adenovirus.

Notch effectors can also comprise or be linked to a transduction domain that enables the Notch effector to cross the plasma membrane of the cells. In such embodiments, the transduction domain enables the effector to cross the plasma membrane and enter the cell from culture medium. Further details of the effectors and domains are given below.

Notch signalling can also be activated by co-culture with cells that express a Notch ligand, e.g. feeder cells expressing Delta or Jagged. Cells can be engineered to express a Notch ligand and then used for this purpose.

Notch signalling can be achieved by coating a portion of apparatus used in cell culture with a Notch ligand. For example a culture plate or dish, or a bead present in the culture can have a Notch ligand applied to its surface.

In embodiments of the invention in which a Notch activator is expressed in a cell, this expression can be constitutive or conditional. Expression can be controlled using recombinase proteins, the Cre-Lox system for example.

Activation of Notch can, as mentioned, be carried out in conjunction with other steps to promote neural specification. Prior to, at the same time as or subsequent to Notch activation, the cells can be induced to differentiate towards a neural fate.

This induction can be achieved by change in the medium in which cells are cultured. In one example, the inducing comprises removing serum from medium in which the cells are being cultured or transferring cells into medium free of serum.

The induction can be achieved by addition or removal of exogenous factors. In one example the inducing comprises adding a factor which promotes neural specification to medium in which the cells are being cultured or transferring the cells into medium containing such a factor. Suitable factors include retinoic acid and BMP antagonists e.g. noggin.

The induction can be achieved through alteration of the physical nature of the culture. For example, the inducing may comprise transferring the cells to a monolayer culture, transferring cells to serum free culture and/or plating on a PA6 feeder layer.

Once neural specification has been commenced it is preferred to take steps to purify the resulting culture, so as to increase further the proportion of neural cells. Another property of Notch activation can thus be exploited in the invention as Notch signalling has been found also to prevent or reduce differentiation of neural progenitors. By maintaining activation of Notch signalling, cells can be maintained as neural progenitors for longer periods. Maintenance of Notch activation can be achieved by continuing to maintain Notch ligands in the medium or continuing to express a Notch activator.

Further improvement of the proportion of neural cells is suitably carried out by removal of non-neural cells. In particular, multipotent or pluripotent cells which have not differentiated may need to be removed. One method is to culture the cells in, or transfer the cells to, medium or culture conditions which are non-permissive for the multipotent or pluripotent cells. In this way, Notch signalling promotes neural specification and non-permissive conditions remove the starting multi- or pluripotent cells.

Generally, absence of LIF is non-permissive for mouse pluripotent cells. Addition of a reducing agent into the culture is also generally non-permissive for pluripotent cells—for example mercaptoethanol can be added to the culture medium. If cells are replated at lower density this reduces the level of autocrine LIF and is also non-permissive for pluripotent cell survival.

The cultures may alternatively or additionally be improved using preferential expression in the desired neural cells of a selectable marker. A method of purifying the cells comprises expressing in the neural progenitor cells a selectable marker and selecting for cells expressing the marker. The selectable marker may encode resistance to antibiotic, in which case antibiotic can be used selectively to deplete the culture of non-neural cells, or a cell surface marker or a fluorescent protein, in which case antibodies to the marker or FACS can be used. Other selection strategies are known and described e.g. in EP 0695351.

As with other aspects of the invention, cells used in the methods are suitably pluripotent cells, especially ES cells, and separately can be rodent or primate cells, especially mouse or human cells. It is particularly preferred that the cells obtained are human neural or neural progenitor cells, obtained from human pluripotent cells.

In an example of operating within the invention, ES cells were differentiated in the presence of Notch signalling, leading to a mixed culture of ES (i.e. cells which have not differentiated) and NS cells, with a NS proportion of about 90%, of which 60% are radial glial cells and 30% are sox1 positive. This mixed culture was then transferred to medium which does not support ES cell growth or self renewal.

The invention thus uses Notch signalling to improve control of differentiation of pluripotent cells in culture. One approach is to express an activated Notch receptor. Another approach uses a ligand for cell surface receptors. A further approach is simply to add to the culture medium one or more ligands that bind on or outside and activate Notch inside the cells. Most currently known ligands do not normally function as secreted soluble proteins, so these ligands are preferably combined with a transduction domain, e.g. a soluble version of the Notch intracellular domain fused to that peptide, which can cross the plasma membrane of cells when added to culture medium. This circumvents the need for activation/cleavage of endogenous Notch receptor. This approach has been used successfully with cre recombinase and HoxB4 and is well suited to neural induction systems of the invention in which serum (which can inhibit that fusion protein translocation across membranes) is absent. Hes5 overexpression shows similar effects to Notch intracellular domain overexpression, and hence another specific embodiment of the invention is a that-Hes5 fusion, and this is another media additive. Hes5, when over-expressed, has a similar effect to Notch. Other ligands suitable for use in Notch activation include generally the Hes family, e.g. Hes1, Hes5, Hes6 and the Hey genes.

In examples set out in detail below we have used a gain of function approach to show that whilst Notch does not interfere with the self-renewal of embryonic stem cells under expansion conditions (LIF+serum) it does bias cells towards specification into the neural lineage after withdrawal of LIF+Serum. Constitutive expression of an activated form of Notch brought about rapid and synchronous neural specification whilst blocking differentiation into non-neural lineages. Furthermore, activated Notch did not bring about terminal differentiation of ES-derived cells, but rather allowed for their expansion as neural progenitors.

Our data suggest that Notch signalling may be a limiting requirement for neural specification, which is only received by a subset of cells during standard neural differentiation protocols. According to the invention, increasing the number of cells that receive a Notch signal improves the efficiency of neural specification protocols without compromising the ability to expand cells either as ES cells or as neural progenitors.

The invention has used both genetic and non-genetic means of activating Notch signalling pathways. Delta-Fc, Jagged-Fc and Contactin-Fc can also be used to activate Notch, mimicking the effects seen when miss-expressing activated Notch (NotchIC). There are advantages of avoiding resorting to genetic manipulation and we believe that non-genetic techniques will be favoured in future.

In examples set out in more detail below, we miss-expressed an activated form of the Notch receptor. Using constitutively active Notch, ES cells can be maintained in culture as ES cells, though with reduced conversion to non-neural cells. If the medium is then changed from LIF+BMP or LIF+serum to a N2B27-based medium, for differentiation, with Notch continuing to be expressed then the cells more rapidly and at a higher % than previously convert to neural cells. The cells become sox1 positive and then BLBP positive and can be kept as BLBP positive cells for several weeks. Some neurons are obtained which don't proliferate. When BMP is added there is no non-neural differentiation (whereas BMP would hitherto have been expected to drive non-neural differentiation).

The invention brings with it a number of advantages. There is an increased proportion of neural cells and a decreased proportion of non-neural. The contaminating cells tend to be just ES cells, which can be removed by transfer to non-ES supporting medium or adopting non-ES permissive conditions and/or media. We have additionally found activation of Notch signalling to be straightforward to do using both genetic manipulation and external ligands.

Notch activation has been found not to have a detrimental effect on ES cell self renewal and propagation. This is an advantage as pluripotent cells in which Notch is activated can be cultured as before and then when induced to differentiate Notch activation is used to reduce contamination of the desired neural culture by non-neural cells.

Increased cell density can be achieved and the cultures have been found to be more robust.

Further aspects of the invention provide medium additives, media and nucleotides and vectors encoding certain additives.

A nucleotide sequence of the invention encodes an activated form of a Notch receptor. The activated form of a Notch receptor preferably comprises an intracellular domain of a Notch receptor. This sequence can be used to provide Notch signalling without using external ligands and can be expressed reversibly or constitutively in cells so as to provide continuous Notch signalling. Also preferably, the activated form of a Notch receptor lacks an extracellular domain of a Notch receptor.

A vector of the invention comprises this nucleotide sequence. The vector of an embodiment of the invention used in examples below comprises a promoter that expresses the activated form of a Notch receptor in pluripotent cells and neural cells. The vector is used to transform cells, e.g. pluripotent cells so as to express the activated receptor.

A further nucleotide sequence of the invention encodes a downstream effector of Notch signalling. The effector is preferably selected from the Hes and Hey transcription factors. This sequence can also be used to provide Notch signalling without using external ligands and can be expressed reversibly or constitutively in cells so as to provide continuous Notch signalling.

A further vector of the invention comprises this nucleotide sequence. The vector of an embodiment of the invention used in examples below comprises a promoter that expresses the effector in pluripotent cells and neural cells. The vector is used to transform cells, e.g. pluripotent cells so as to express the effector.

The invention also provides a composition comprising a downstream effector of Notch signalling or an activated form of a Notch receptor and a transduction domain. This composition can be added to or included in culture medium so as to provide an activator of Notch signalling. The activated form of a Notch receptor typically comprises an intracellular domain of a Notch receptor.

The transduction domain enables the composition to enter cells. A number of suitable transduction domains are known in the art and reference to a transduction domain or a translation domain refers to a domain or fragment of a protein which effects transport of itself and/or other proteins and substances across a membrane or lipid bilayer and encompasses native domains and fragments, variants and derivatives that retain this binding function. The latter membrane may be that of an endosome where translocation will occur during the process of receptor-mediated endocytosis. Translocation domains can frequently be identified by the property of being able to form measurable pores in lipid membranes at low pH (Shone et al. (1987) Eur J. Biochem. 167, 175-180 describes a suitable test). The latter property of translocation domains may thus be used to identify other protein domains which could function as the translocation domain within the construct of the invention. Examples of translocation domains derived from bacterial neurotoxins are as follows:
Botulinum type A neurotoxin—amino acid residues (449-871)
Botulinum type B neurotoxin—amino acid residues (441-858)
Botulinum type C neurotoxin—amino acid residues (442-866)
Botulinum type D neurotoxin—amino acid residues (446-862)
Botulinum type E neurotoxin—amino acid residues (423-845)
Botulinum type F neurotoxin—amino acid residues (440-864)
Botulinum type G neurotoxin—amino acid residues (442-863)
Tetanus neurotoxin—amino acid residues (458-879)

Other suitable translocation domains are TAT (e.g. from HIV-1) and penetratin, short sequences of amino acids that internalize covalently linked peptides and convey them, or enable them to be conveyed, to the nucleus. Further suitable domains, referred to as protein transduction domains, such as VP22, derivatives of antennapedia and others, are described in Wadia et al, 2002. These domains can be linked to a Notch ligand or activated form of a Notch receptor chemically, e.g. via thiol functional groups or a fusion can be expressed comprising both components. The linked molecules, the fusions and compositions comprising the same form other aspects of the invention. These can be used e.g. as additives to culture medium as an alternative to transfecting cells with Notch ligands or activated forms of Notch receptors.

“Translocation” in relation to translocation domain, means the internalization events which occur after binding to the cell surface. These events lead to the transport of substances into the cytosol of cells.

A composition for delivery of a Notch effector or an activated form of a Notch receptor 10 to an ES cell therefore comprises:
the Notch effector or the activated form of a Notch receptor, and
a translocation domain that translocates the Notch effector or the activated form of a Notch receptor into the ES cell.

The translocation domain can also be selected from (1) a H_{N} domain of a diphtheria toxin, (2) a fragment or derivative of (1) that substantially retains the translocating activity of the H_{N} domain of a diphtheria toxin, (3) a fusogenic peptide, (4) a membrane disrupting peptide, and (5) translocating fragments and derivatives of (3) and (4).

Further provided by the invention are isolated nucleotides encoding the fusion proteins of the invention and vectors comprising these nucleotides.

A medium of the invention, for culture of neural cells, comprises a component which activates Notch signalling in cells in the medium.

The medium may comprise a Notch ligand, which can be a multivalent Notch ligand and capable on its own of clustering Notch receptors. The medium may contain a Notch ligand-tag fusion protein and a clustering molecule which binds to two or more such fusion proteins. The medium may comprise an activated Notch receptor, such as an intracellular domain of a Notch receptor, or a Notch effector, optionally linked to a transduction domain as described above.

Further medium of the invention may be non-permissive for multipotent or pluripotent cells. This medium can be used to further deplete the culture of cells which have not differentiated into neural cells. Preferred medium is non-permissive for pluripotent cells.

A still further aspect of the invention provides a method of culture of neural cells to as to increase the density of cells in culture, the method comprising activating Notch signalling in the cells. Notch activation can be carried out as described for all other aspects of the invention. We have found that, in the absence of Notch activation, neural induction declines at cell densities 10⁴ cells per cm² but that in the presence of Notch activation in accordance with the invention neural induction can be successfully be achieved at densities of 5×10⁴ cells per cm² and that the resultant cultures are more resistant to small changes in culture conditions and are hence regarded as more robust cultures.

Another aspect of the invention provides a method of culture of pluripotent cells, preferably ES cells, so as to maintain the cells in a self-renewing state, comprising culturing the cells in medium comprising an agonist of a BMP receptor and in the presence of Notch activation. BMP agonists are suitably BMP 2 and BMP 4. Notch activation is preferably as described herein in respect of the other aspects of the invention.

A still further aspect of the invention provides a pluripotent cell in which Notch signalling has been activated by any of the embodiments of the invention. The cell is preferably a mouse or human cell and preferably an ES cell. A specific embodiment of this aspect of the invention is an ES cell engineered to express a peptide comprising a Notch intracellular domain.

The invention is now described in specific examples, illustrated by drawings in which:

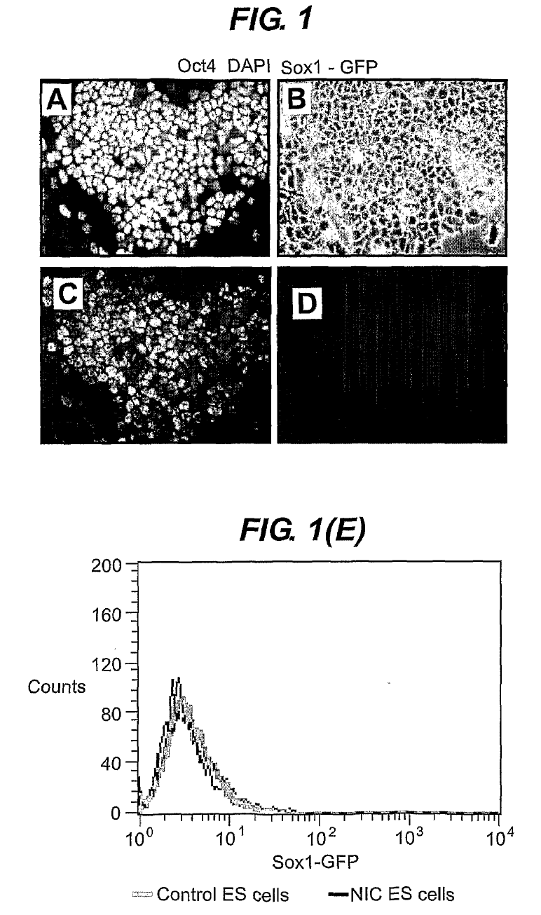

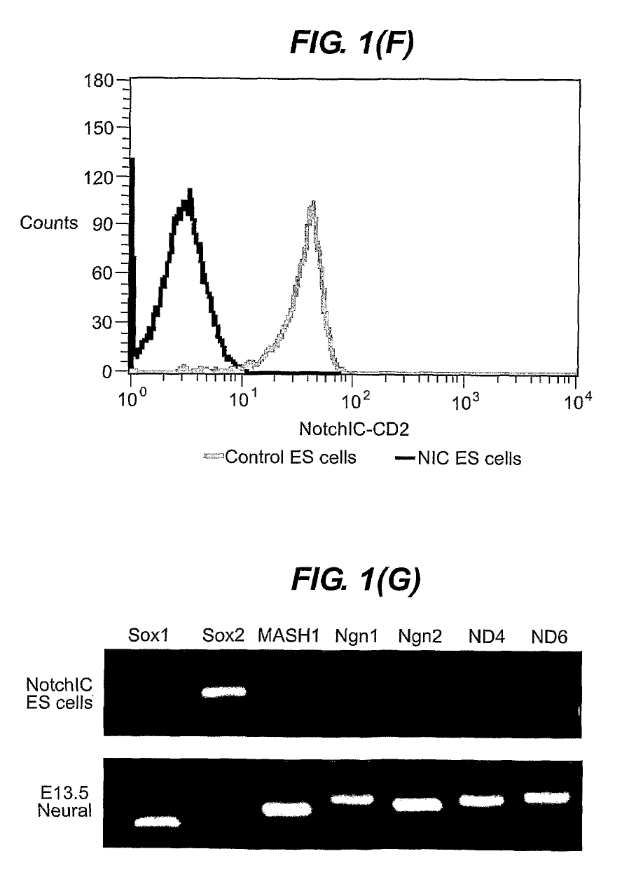

FIG. 1 shows NotchIC ES cells or parental control 46C ES cells maintained in LIF+serum unless otherwise stated;

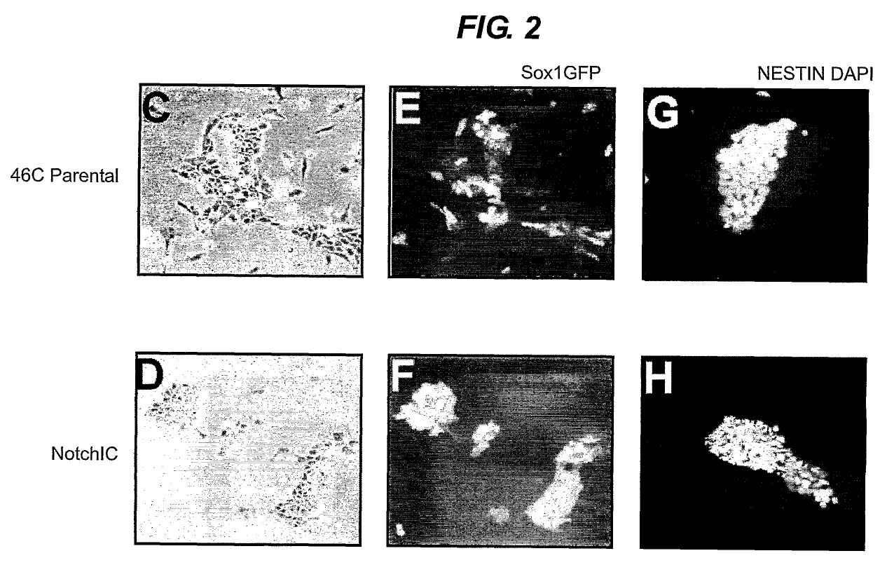

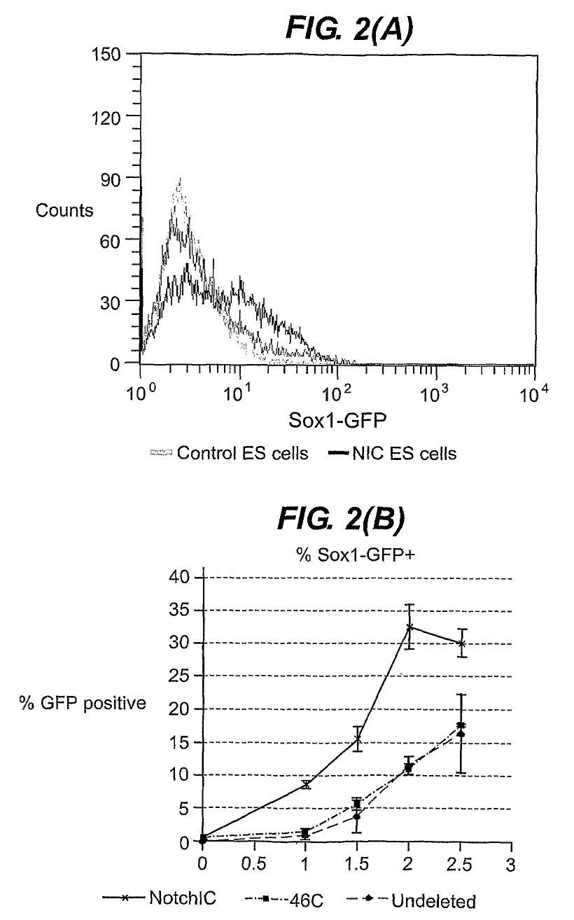

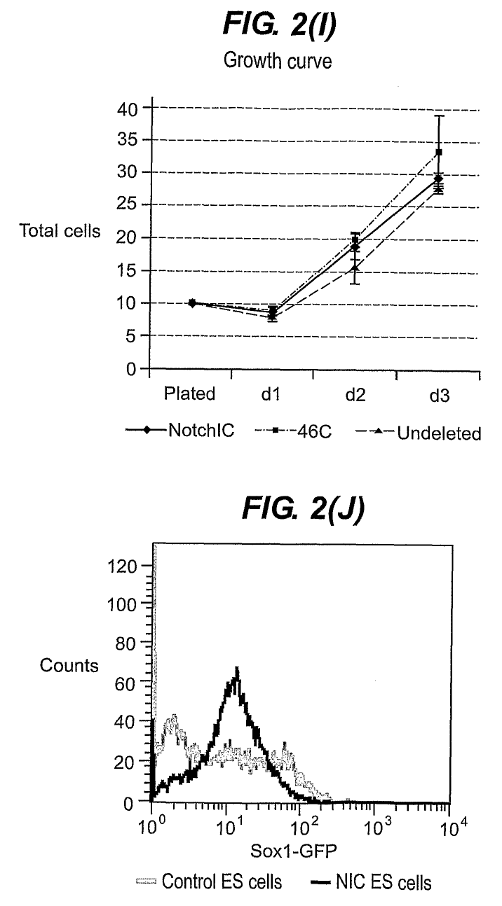

FIGS. 2-9 show NotchIC ES cells or parental control 46C cells cultured under monolayer differentiation conditions and the results of analysis of those cultures; and

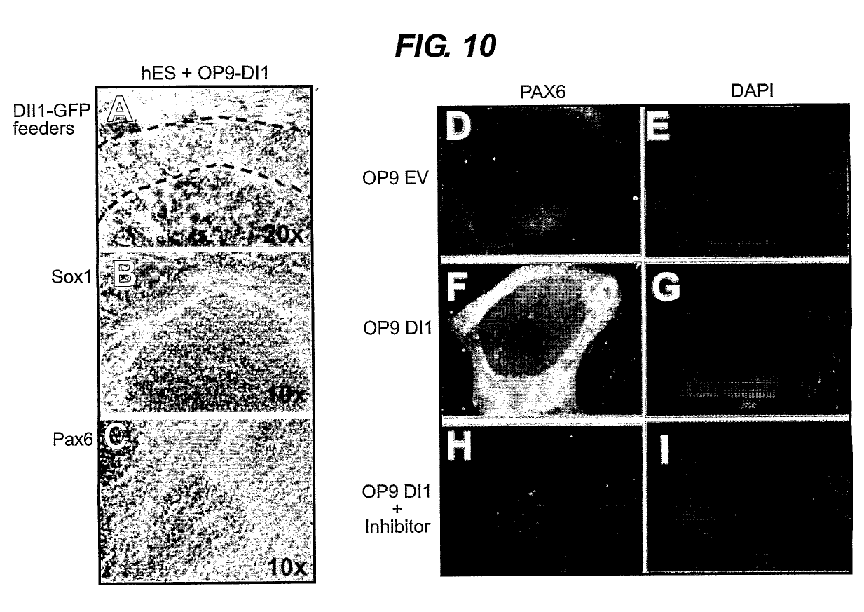

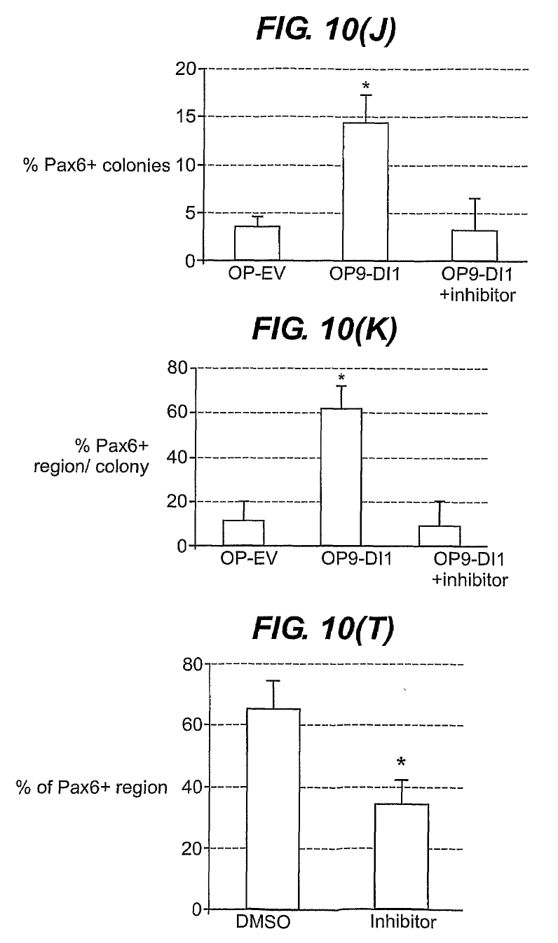

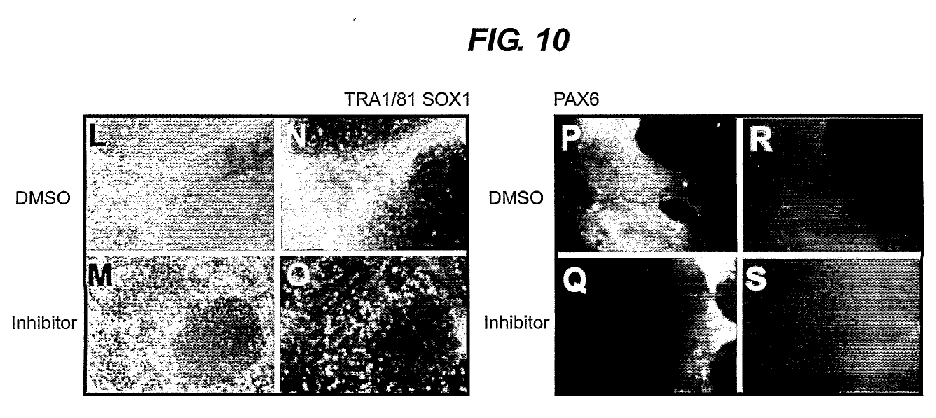

FIG. 10 shows human ES cells cultured on feeder cells or under monolayer differentiation conditions and the results of analysis of those cultures.

In more detail:

FIG. 1 shows NotchIC ES cells or parental control 46C ES cells maintained in LIF+serum unless otherwise stated. (A-D) Colonies of NotchIC ES cells shown under phase contrast or stained for markers as indicated. E: Populations of NotchIC ES cells or parental control ES cells analysed by FACS passage 12 for expression of sox1-GFP. F: Populations of NotchIC ES cells or parental control ES cells analysed by FACS passage 12 for expression of NotchIC-CD2. G: RT-PCR analysis of Notch IC cells cultured in LIF+Serum (NotchIC ES cells) and of E13.5 embryo neural tissue as a positive control for neural markers.

FIG. 2 shows NotchIC ES cells or parental control 46C cells cultured under monolayer differentiation conditions. A: Typical FACS profile of sox1-GFP expression after 48h. B: Graph to indicate results of FACS analysis of the proportion of sox1-GFP positive cells at various time points from triplicate cultures. C-H: Intact cultures at 72h of monolayer differentiation, shown in phase contrast or stained for markers as indicated. I: Growth curve indicates the total number of cells at various time points in triplicate cultures. J: Typical FACS profile of sox1-GFP expression after 5 days.

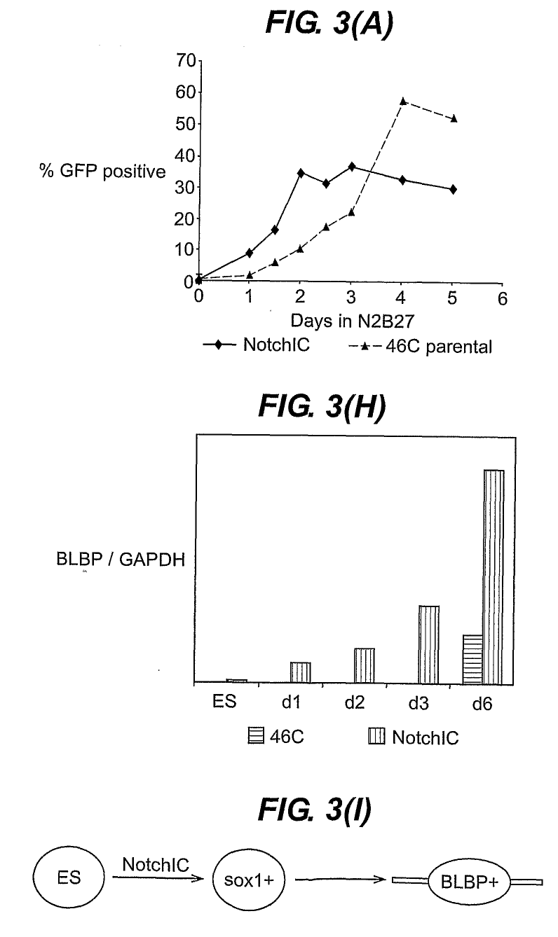

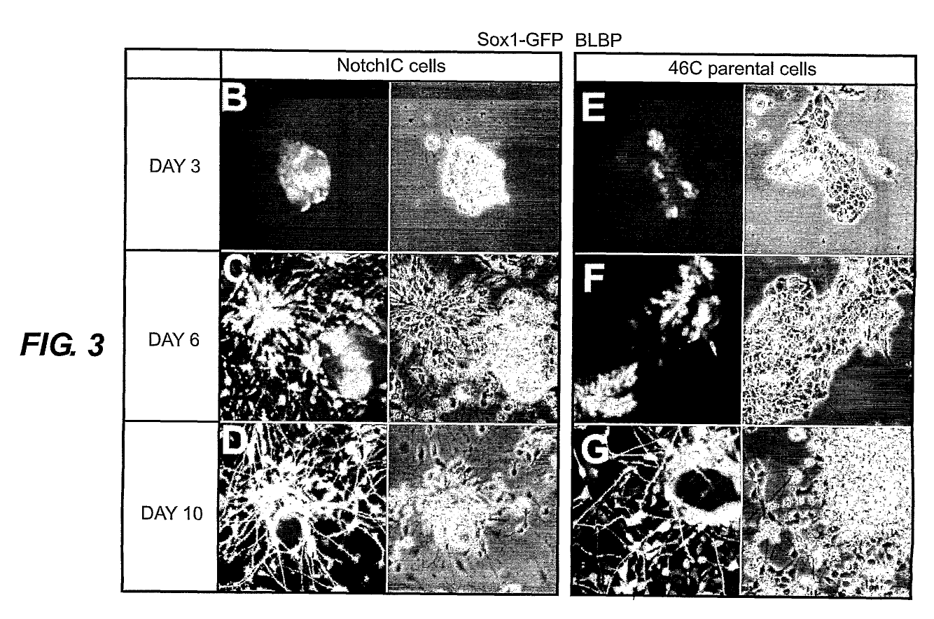

FIG. 3 shows NotchIC ES cells or parental control 46C cells cultured under monolayer differentiation conditions. A: Graph to indicate results of FACS analysis of the proportion of sox1-GFP positive cells at various time points. B-G: Intact cultures at various time points shown in phase contrast or stained for markers as indicated. H: Quantitative PCR for BLBP during monolayer differentiation. I: Schematic diagram to illustrate the transition of ES cells into sox1-GFP positive neuroepithelial progenitors and then into BLBP+ radial glial neural progenitors.

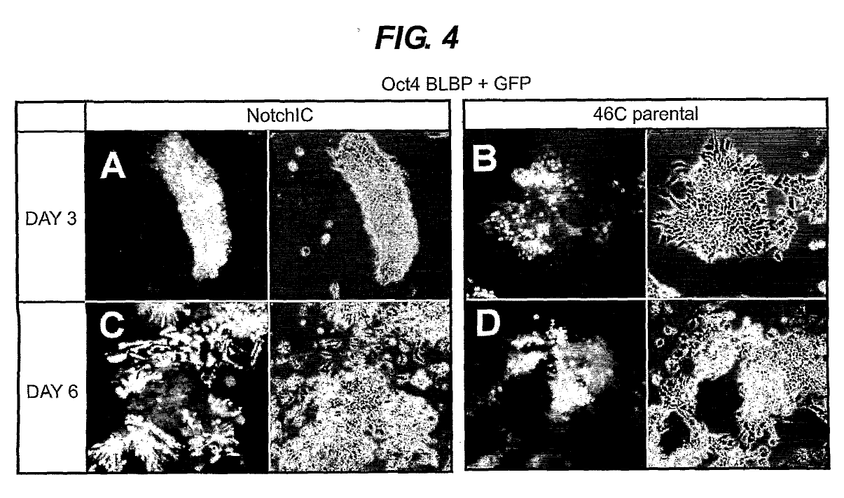

FIG. 4 shows NotchIC ES cells or parental control 46C cells cultured under monolayer differentiation conditions and stained for Oct4 (red) to indicate ES cells together with a combination of BLBP and GFP (green) to indicate both types of neural progenitor together.

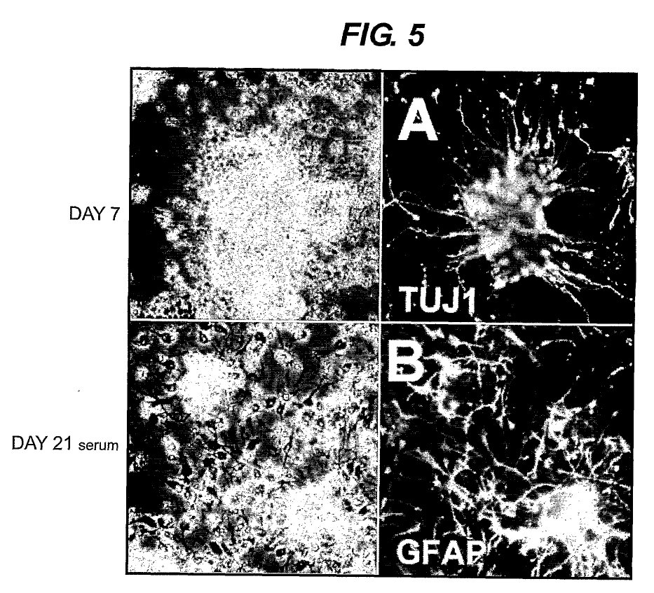

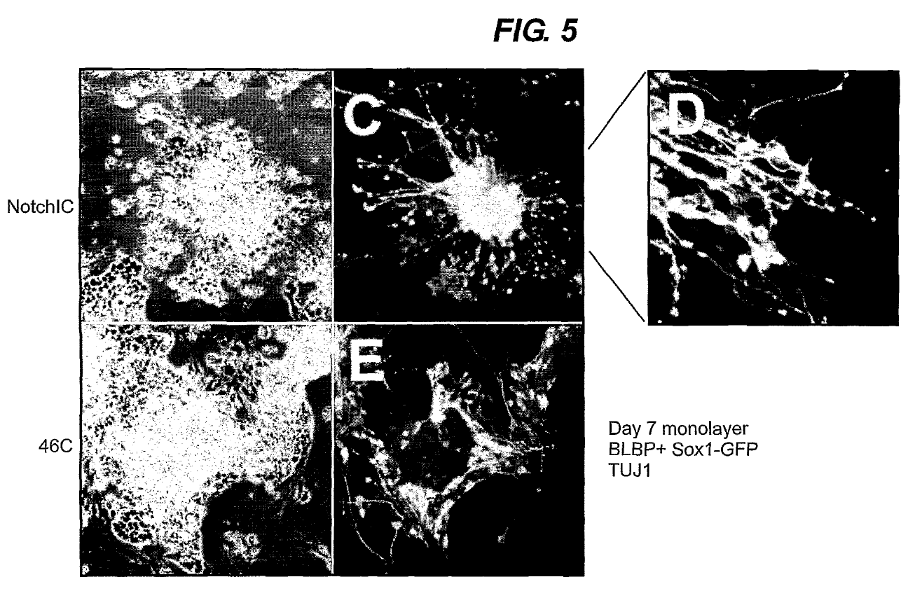

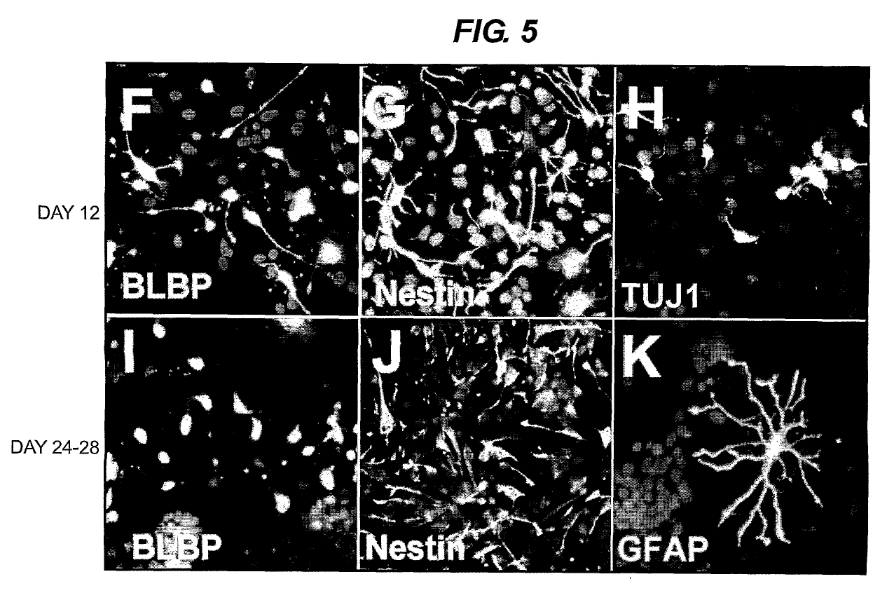

In FIG. 5, A, C, D are intact cultures at day 7 of monolayer differentiation, shown in phase contrast or stained for markers as indicated. B: Cells replated onto gelatin after 7 days monolayer differentiation, cultured for a further 7 days in the absence of serum then for the final 7 days in the presence of serum and 100 units/ml LIF, shown in phase contrast or stained for GFAP. F-K: Cells replated onto laminin after 7 days of monolayer differentiation and cultured for a further 5 days (F,G,H: total 12 days), 16 days (J: total 24 days) or 21 days (K: total 28 days) then fixed and stained for markers as indicated.

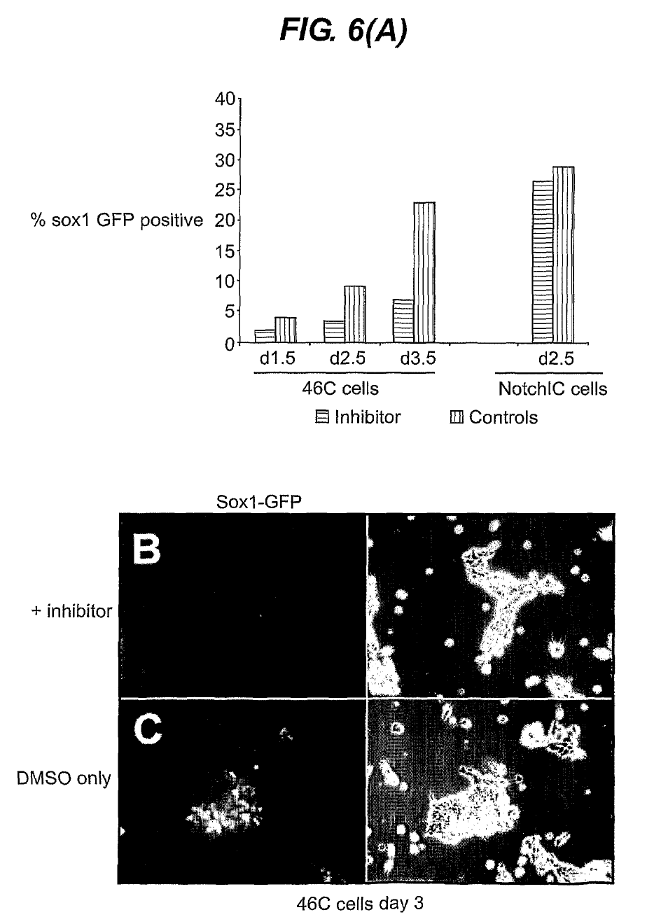

FIG. 6 shows the proportion of sox1-GFP cells (A) or GFP expression within intact cultures (B, C) after monolayer differentiation of 46C cells or NotchIC cells exposed to 4 uM gamma secretase inhibitor or to equivalent amounts of DMSO diluent.

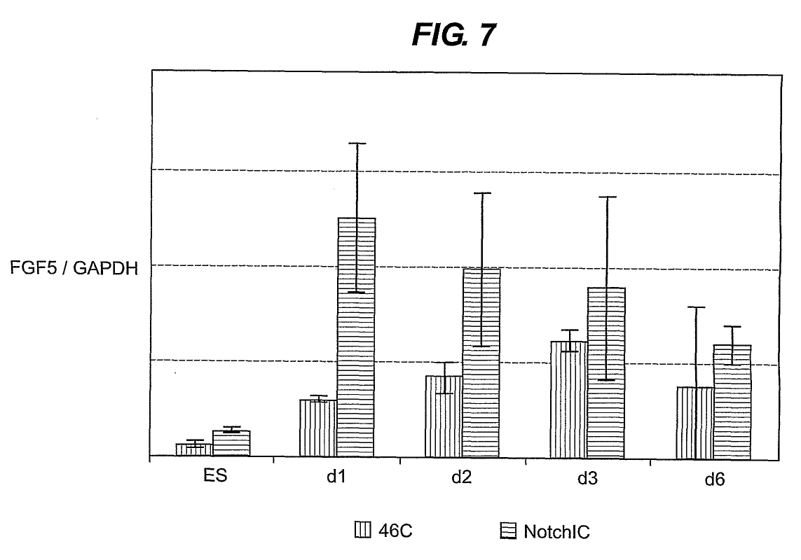

FIG. 7 shows quantitative PCR for FGF5 during monolayer differentiation.

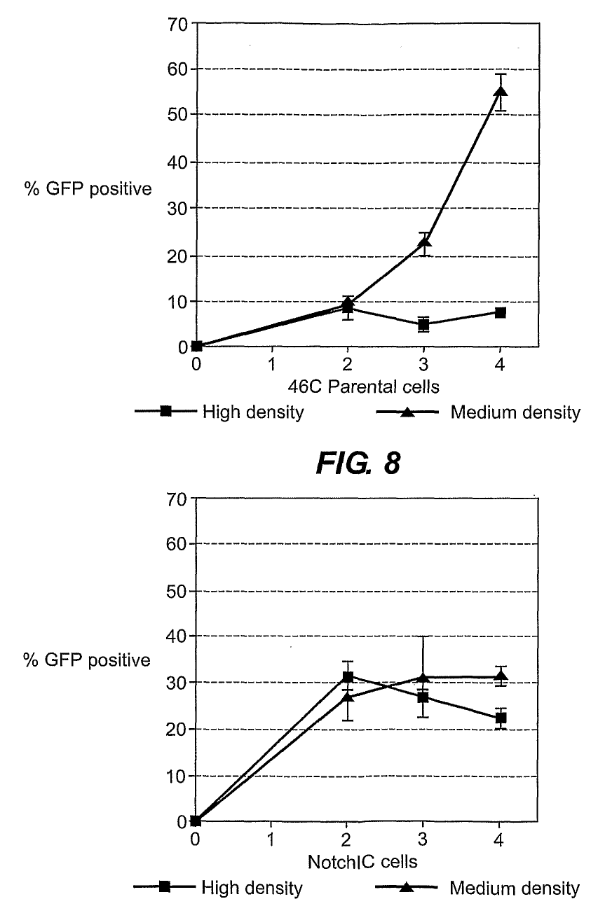

FIG. 8 shows FACS plots indicating the proportion of sox1-positive cells after monolayer culture of NotchIC cells or parental control cells at medium (10⁴ cells/cm²) or higher (3×10⁴ cells/cm²) densities.

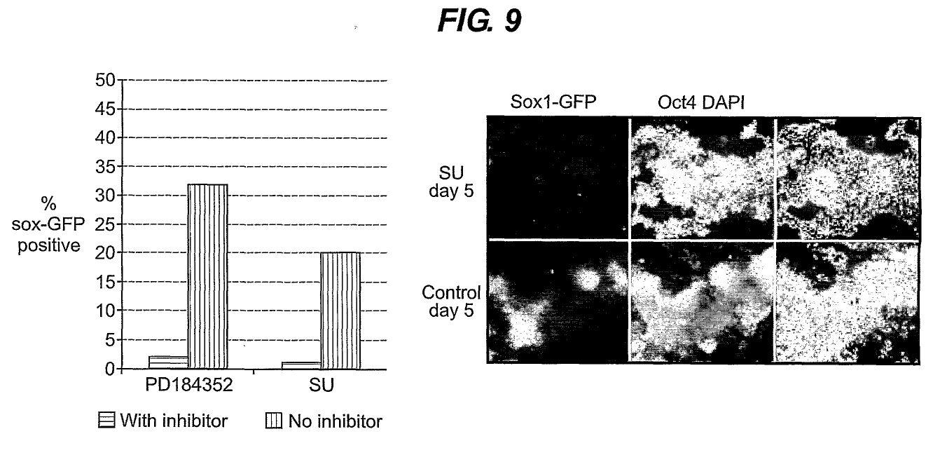

FIG. 9 shows the results when NotchIC cells or parental control 46C cells were cultured for 3 days in the presence of 4 uM PD compound, SU compound, or in an equivalent concentration of DMSO diluent (‘No inhibitor”) and the proportion of sox1-GFP positive cells analysed by FACS on day3. Intact cultures were stained for Oct4 on day 5 to visualise undifferentiated ES cells.

FIG. 10 shows (A-I) Human ES cells plated on OP9 feeder cells expressing either GFP only (OP9 EV) of the Notch ligand Delta1 (OP9 D11) with γ-secretase inhibitor where indicated (“+inhibitor) were cultured for 7 d and stained for markers as indicated. (A) Higher magnification picture to indicate the cell morphology. (J,K) Quantification of Pax6 immunostaining (averages and standard eviations shown from four experiments). (L-T) Human ES cells grown under mono-layer differentiation conditions in the presence of γ-secretase inhibitor (“inhibitor”) or DMSO vehicle and stained for Pax6,Sox1, or TRA1/81 as indicated. T shows quantification of Pax6 immunostaining (averages and standard deviations from four experiments,

Specific embodiments of the invention provide or use one or more of the following sequences, referred to herein by their SEQ ID NO:

| SEQ ID NO | Description |
|---|---|
| 1 | Notch full length DNA |
| 2 | Notch full length amino acid |
| 3 | Notch intracellular DNA |
| 4 | Notch intracellular amino acid |
| 5 | Hey1 DNA |
| 6 | Hey1 amino acid |
| 7 | Hey2 DNA |
| 8 | Hey2 amino acid |
| 9 | Hes1 DNA |
| 10 | Hes1 amino acid |
| 11 | Hes3 DNA |
| 12 | Hes3 amino acid |
| 13 | Hes5 DNA |
| 14 | Hes5 amino acid |
| 15 | Hes6 DNA |
| 16 | Hes6 amino acid |
| 17 | Fusion of NotchIC-tat |
| 18 | Fusion of NotchIC-protein |
| | transduction domain from |
| | antennapedia |
| 19 | Fusion of tat-Hey2 |
| 20 | Fusion of tat-Hes5 |

### EXAMPLES

Materials and Methods

Targeting NotchIC into 46C ES cells

Our targeting construct was based on similar constructs previously used for targeting into the ROSA locus. It contains a pgk-neo cassette flanked by loxP sites, followed by the coding sequence for the intracellular domain of NotchIC (Kopan et al., 1994) followed by an internal ribosomal entry site followed by the coding sequence for the human cell surface molecule CD2. This construct was transfected into 46C ES cells (Ying et al., 2003b) by electroporation, and clones were expanded under G418 selection. Correctly targeted clones were identified by Southern blotting after digestion with EcoRV. DNA gives an 11 kb band whilst untargeted wild type cells give a 3.8 kb fragment. A clone of targeted cells were transfected with a plasmid containing CRE under the control of the pCAG promoter.

ES cell culture

ES cells were maintained in GMEM supplemented with 2-mercaptoethanol, non-essential amino acids, sodium bicarbonate, 10% fetal calf serum (FCS) and 100 units/ml LIF on gelatinised tissue culture flasks (Smith, 1991).

Monolayer Differentiation

This is as described in detail in Ying et al., 2003b. Briefly, ES cells were washed to remove all traces of serum and then plated on gelatin-coated tissue culture plastic at a density of 1>10⁴ cells/cm² in N2B27 serum-free medium. N2B27 consists of a 1:1 ratio of DM/F12 and Neurobasal media supplemental with 0.5% N2 (made in house as described in (Ying et al., 2003b)), 0.5% B27 (Gibco) and 2-mercaptoethanol. Media was changed every second day.

In some experiments, the culture medium was supplemented with 100 units/ml LIF and with 10% FCS. The MAPK inhibitor PD184352 (gift of P. Cohen, Univ Dundee) was used at a concentration of 4 μM. The gamma secretase inhibitor (Calbiochem cat. 565771) was used at a concentration of 4 μM. Neither of these inhibitors had any obvious toxic effects over the time course of the experiments.

Immunofluorescence

Cells were fixed in 4% paraformaldehyde and incubated for 30 minutes in blocking buffer (PBS, 2% Goat serum and 0.1% Triton). Primary antibodies were diluted in blocking buffer and applied for 1 h at room temperature. After three washes in PBS, secondary antibodies conjugated to Alexa fluorophores (Molecular Probes) were diluted at 1:1000 in blocking buffer and applied for 1 h at room temperature. The cells were washed at least three times in PBS and visualised on a Olympus inverted fluorescence microscope.

In experiments where cells were counted, nuclei were counterstained with DAPI and at least 1000/cells per culture were counted from three separate cultures and an average taken.

Primary antibodies were obtained from the following sources:

Human CD2 (BD Biosciences); Oct4 (Santa Cruz); GFP (Molecular Probes); Nestin (DSHB); BLBP (Gift); Neuronal beta-III tubulin (Covance); GFAP (Sigma); O4 (DSHB); RC2 (DSHB). Antibodies obtained from the Developmental Studies Hybridoma Bank were developed under the auspices of the NICHD and maintained by The University of Iowa, Department of Biological Sciences, Iowa City, Iowa 52242.

Human ES Cell Culture and Differentiation

Undifferentiated human ES cells were maintained on a layer of human foreskin fibroblast (ATCC, Manassas, Va., United States) in the defined medium N2B27 supplemented with LIF (10 ng/ml), BMP-4 (3 ng/ml; R & D Systems, Minneapolis, Minn., United States), and bFGF (10 ng/ml; R & D Systems). Cells were passaged at a split ratio of 1:2 every week using collagenase IV (1 mg/ml). Feeder-free neural differentiation was performed following the monolayer protocol used for mouse cells, modified to suit human ES cells as follows: cells reaching 50% confluence were incubated in collagenase IV for 15 min, washed once in PBS and detached in N2B27 medium supplemented with bFGF (FGF medium, no LIF, no BMP4) using glass beads. Cells were then incubated for 4 h in a gelatinised flask in the same medium to allow the differential attachment of the feeder cells. Finally, the ES suspension was plated at a 1:1 ratio in culture dishes pre-coated with Matrigel (low growth factor Matrigel, 1:20; BD Biosciences PharMingen, San Diego, Calif., United States). When indicated, the γ-secretase inhibitor (4 μM) was added from the start of the feeder-removal step and then added every other day when the medium was changed. For coculture with OP-9 cells, hES cells were treated with collagenase as described above and then manually detached to avoid carry-over of human fibroblasts. Cells were then directly plated in FGF medium on a layer of γ-irradiated OP9-EV or OP9-D11 stromal cells (kindly provided by A. Cumano). Matrigel was used to promote the survival of the OP9 in the serum-free medium. The γ-secretase inhibitor was added at plating and then added every other day when the medium was changed.

Quantification of Neural Differentiation of Human ES Cells.

Cells were processed for immunocytochemistry and neural differentiation quantified as follows. For OP9 coculture experiments, the number of colonies with positive PAX-6 cells was counted and normalised to the total number of colonies in the well. For all experiments (feeder-free and OP9-dependent differentiation), Velocity image analysis software (Improvision, Lexington, Mass., United States) was used to quantify the extent of differentiation. Briefly, the software was used to calculate the area of the well (feeder-free experiments) or of each colony (OP9 experiments) covered by PAX-6-positive nuclei. The values were then normalised to the area covered by the cells or colonies, using DAPI staining. All experiments were repeated at least three times with four wells per condition.

### Example 1-1

ES Cells and Early Neural Progenitors Express Notch Receptors and Ligands

We used RT-PCR to confirm that Notch1 and Jagged2 are expressed in ES cells and their earliest neural derivatives.

### Example 1-2

Generation of 46C Cells Constitutively Expressing NotchIC

We generated a targeting construct in which a sequence encoding the intracellular domain of Notch (Kopan et al., 1994) was preceded by a floxed stop-pgk-neo cassette, and followed by a sequence encoding IRES-human CD2. Human CD2 is a cell surface molecule with no phenotypic effect on mouse cells, used here as tag to indicate NotchIC missexpression. This construct was targeted into the ROSA locus of 46C cells. 46C cells are a line of ES cells that contain the coding sequence of GFP targeted into one allele of the early neural marker sox1, and thus act as a convenient experimental system for monitoring neural induction [Stavridis, 2003 #221].

A clonal line of NotchIC-targeted ES cells was transfected with CRE in order to excise the stop codon and activate constitutive transcription of NotchIC-IRES-CD2. The successfully deleted population (designated “NotchIC cells”) was separated from the undeleted population by FACS based on CD2 expression. The undeleted population was used as a control and these are referred to as “undeleted controls”. Parental 46C cells (not targeted with NotchIC) were used as a second control population, and these are referred to as “parental 46C controls”. Both control populations gave similar results in all experiments.

### Example 1-3

NotchIC ES Cells are Indistinguishable from Control ES Cells

Notch IC cells showed no difference in growth rate or morphology compared to either undeleted controls or parental 46C control ES cells under standard culture conditions for maintaining undifferentiated proliferative ES cells (LIF and serum). They grew at a comparable rate over more than 20 passages (data not shown) expressed markers of undifferentiated ES cells, and lacked markers of differentiation (FIG. 1 and data not shown).

### Example 2-1

Populations of NotchIC Cells Undergo Neural Specification More Rapidly and Homogenously than Controls

We next tested the effect of NotchIC on neural specification. We transferred NotchIC or control ES cells into a neural differentiation protocol that is based on adherent monolayer culture in the absence of exogenous growth factors (Ying and Smith, 2003). Cells were disaggregated and analysed by FACS for sox1-GFP expression every 12 hours during monolayer differentiation. Control ES cells generate sox1-GFP+ cells gradually: fewer than 2% could be detected after 24 h, and only 1111% after 48 h, In contrast, NotchIC cultures contained 9±1% sox1-GFP+ cells after only 24 h, increasing to 32±3% by the second day (FIG. 2A, B).

The distribution of sox1-GFP+ cells was also monitored in intact monolayer differentiation cultures, using an inverted fluorescence microscope. Cultures of control ES cells contain GFP-positive cells interspersed with GFP-negative cells in a “salt and pepper” pattern (FIG. 2C,E). In NotchIC cultures, distribution of GFP+ cells is more homogenous (FIG. 2D,F). There is also a difference in the variability of GFP intensity between cells: NotchIC cells have uniformly weak GFP expression whilst control cultures contain a mixture of bright and dim GFP cells (FIG. 2E, F: see also FACS profile J). Staining for another early neural marker, nestin, was consistent with entry of the NotchIC cells into the neural lineage (FIG. 2G, H).

FGF5 is a marker of primitive ectoderm, expressed transiently at an intermediate stage of differentiation of pluripotent ES cells towards neural tissue. Control ES cells acquire FGF5 at increasing levels over the first few days of monolayer differentiation, after which it declines as Sox1 expression increases (FIG. 7). In contrast, NotchIC-overexpressing ES cells have a maximal peak of high FGF5 expression after just 24 h differentiation, in keeping with the rapid induction of sox1in these cells (FIG. 7). This data also shows that NotchIC promotes the transition of ES cells into the neural lineage.

### Example 2-2

NotchIC does not Increase Proliferation

The high proportion of sox1-GFP+ cells that emerge early during monolayer differentiation of NotchIC ES cells could be explained by an increase in the rate of conversion of ES cells to sox1+neural cells. Alternatively, it could be that NotchIC does not affect neural specification but instead increases the rate of proliferation in neural cells. We consider that the second possibility is unlikely because there are significant numbers of sox1-GFP+ cells even after only 24 h, before the onset of neural specification in control ES cell populations (FIG. 2B). Furthermore, the growth rate of NotchIC and control populations is indistinguishable during the course of the experiment (FIG. 2I).

### Example 2-3

NotchIC Overcomes the Inhibitory Effects of High Cell Density on Neural Specification of ES Cells

Neural induction of normal ES cells during monolayer differentiation is strongly inhibited by even modest increases in cell density (FIG. 8). We found that NotchIC cells are resistant to the inhibitory effects of increasing cell density on neural differentiation.

### Example 2-4

NotchIC does not Bypass the Requirement for FGF Signalling in Neural Specification

Although differentiation of ES cells into neural progenitors does not require exogenous growth factors, it is dependant upon FGF signalling, which is most likely provided by autocrine FGF4 (Ying and Smith, 2003). We found that NotchIC does not bypass this requirement: the MAPK inhibitor: PD184352 and the FGFR inhibitor SU5402 were both able to block induction of sox1 in NotchIC cells (FIG. 9).

### Example 3-1

Sox1 Induction is Rapidly Followed by BLBP Induction

Sox1-GFP is expressed only transiently during monolayer differentiation of NotchIC cells. The intensity of sox1-GFP within each individual cell does not accumulate to high level in comparison to control cells (FIG. 2: compare E and F; also FIG. 2J), whilst expression within the NotchIC population as a whole reaches a plateau by day 3 and begins to decline soon afterwards (FIG. 3A).

Between day 4 and day 6, the majority of NotchIC cells undergo a striking morphological change to become bipolar (FIG. 3C). Control ES cells only rarely develop this bipolar morphology at this time (FIG. 3F). These bipolar cells express BLBP (FIG. 3 C,D) and RC2 (not shown). Furthermore, we found that expression of BLBP preceded any overt morphological change, appearing as early as the third day of monolayer differentiation (FIG. 3A). By the fifth day, at least 50% of NotchIC cells express BLBP, compared with fewer than 5% of control cells. At this time, there was little or no terminal differentiation into neurons (TUJ1 immunostaining), astrocytes (GFAP immunostaining) or oligodendrocytes (O4 immunostaining) (data not shown).

Quantitative PCR confirmed that BLBP was rapidly induced in NotchIC cells during the first few days of monolayer differentiation, whereas it remained undetectable in control cultures for the first five days (FIG. 3H)

BLBP-positive RC2-positive radial glia are descendants of sox1-positive neuroepithelial cells in vivo. The term glia is misleading: these cells are a major source of both neurons and glia in vivo and should be considered neural progenitors rather than differentiated glial cells. ES-derived neural cells mimic their in-vivo counterparts, progressing over time from an early sox1-postive progenitor to a later BLBP-positive progenitor (Conti, Pollard et al: (Bibel et al., 2004): see also FIG. 3, E, F, G). In keeping with these previous reports, we now find that NotchIC ES-derived early neural cells rapidly transform into BLBP+/neural progenitors, and this explains why numbers of sox1-GFP cells do not continue to increase beyond 3 days in monolayer differentiation.

Sox1-GFP expression is downregulated as BLBP is upregulated, such that the two markers are generally mutually exclusive (although the earliest BLBP+ cells do sometimes coexpress weak levels of GFP, possibly due to perdurance of GFP protein. data not shown). The total number of neural progenitors in day 5 monolayer can therefore be estimated by adding the number of sox1GFP+cells (around 30%) to the number of morphologically mature BLBP+ cells (more than 50%) to give a total of more than 80% neural cells.

FIG. 4 shows typical cultures stained in green for both BLBP and Sox1-GFP in order to visualise all neural progenitors together, counterstained in red for Oct4 to indicate undifferentiated ES cells. In NotchIC cultures, the vast majority of the cells express neural markers by day 3 (FIG. 4A), with the only cells that resist neural differentiation being a minor subpopulation of undifferentiated ES cells (possibly due to autocrine LIF signalling). After three further days of differentiation, it is still the case that practically all cells can be accounted for by expression of either neural or ES markers (FIG. 4C). This contrasts with control cultures, which contain 15-30% of cells that lack both neural and ES cell markers, and which by day 6 have the morphology of non-neural differentiated cell types (FIG. 4B, D). These observations further confirm the rapid and relatively homogenous conversion of ES cells into neural progenitors. They also indicate that Notch biases differentiation in favour of neural and away from non-neural lineages.

### Example 4

NotchIC Allows for the Maintenance of Neural Progenitors Rather than Promoting their Rapid Terminal Differentiation

The Notch signalling pathway has been reported to either promote self-renewal of neural stem cells (Ohtsuka et al., 2001), or to promote their differentiation into astrocytes (Tanigaki et al., 2001), depending upon context in which the gain of function experiments were carried out. In monolayer differentiation cultures, the majority of NotchIC cells persist as BLBP+nestin+RC2+neural progenitors for at least two to three weeks (FIG. 5F, G, H, I, J) (they do not survive much longer than this in the absence of exogenous growth factors). A subpopulation of cells differentiates into neurons during the second week of culture (FIG. 5A,H) but these neurons are outnumbered by undifferentiated BLBP+ cells (FIG. 5C, D, F-H). Astrocyte differentiation occurs only very rarely during the first two weeks (<1%: data not shown). Astrocytes begin to emerge during the third week of serum-free culture, but remain in the minority (FIG. 5K). Oligodendrocytes were never detected. NotchIC did not bring about rapid differentiation into astrocytes or into any other terminally differentiated cell types in our culture system, but rather allowed for the expansion of neural progenitors.

### Example 5

NotchIC Neural Progenitors can Efficiently Differentiate into Astrocytes upon Exposure to Serum

The observation that most NotchIC neural progenitors either differentiate into neurons during the second week, or else remain undifferentiated for at least three weeks, raises the question of whether these cells have significant glial as well as neuronal potential.

In order to address this question, we treated the cells with LIF and serum, which are potent inducers of astrocyte differentiation from late neural progenitors. NotchIC ES-derived neural progenitors, like normal ES-derived neural cells (and similarly to or early embryonic neural cells) remain resistant to this effect of LIF or serum during their first 10-14 days. However, if transferred to serum-containing medium after 14 days than they efficiently (>70%) differentiate into GFAP-positive astrocytes over the subsequent 4 days (FIG. 5B)

The observation that the NotchIC-ES derived cells are able to generate significant numbers of neurons during the first 10 days of serum-free culture, and that they are also able to differentiate into astrocytes with high efficiency after three weeks culture and exposure to serum, confirms that they are neural progenitors.

### Example 6

NotchIC and Neural Specification of ES Cells

A critical step in activating Notch in vivo is its cleavage by gamma secretase to release the intracellular domain. Gamma secretase inhibitors are effective inhibitors of Notch activity. One problem with these inhibitors is that they are not specific to Notch: they also inhibit cleavage by gamma secretase of other molecules. In our experiments we can use NotchIC cells as a negative control for any non-Notch-specific effects of the inhibitor; Since these cells already contain a pre-cleaved NotchIC fragment, they will be immune to effects of the gamma secretase inhibitor on Notch cleavage, whilst remaining vulnerable to any non-notch-specific effects of the inhibitor.

The gamma secretase inhibitor is able to significantly reduce induction of sox1during monolayer differentiation. This appears to be a specific effect on the Notch pathway because there is no significant effect on induction from sox1from cells expressing the constitutively active form of Notch (FIG. 6).

An alternative loss-of-function approach, using mutant ES cellswe have obtained from Tim Schroeder (GSF, Munich) which lack the critical downstream mediator of Notch signalling, RBPJk (Schroeder et al., 2003), is to test whether they fail to differentiate into neural cells and if so whether this can be rescued by transfection with RBPJk plasmid.

### Example 7

Notch Promotes Neural Specification in Human ES Cells

We investigated whether the role of Notch in neural differentiation is conserved in human ES (hES) cells. We first confirmed that the Notch ligands Jagged1, Jagged2, Delta1, and Delta3 could all be readily detected in human ES cells by RT-PCR (FIG. S7). Mis-expression of Notch ligands in feeder cells has been shown to activate Notch in other cell types, so we decided to employ this strategy with hES cells.

We made use of OP9 cells that stably express the Notch ligand Delta1 together with GFP, through retroviral transduction (OP9-Delta1). Control OP9 feeder cells had been transduced with a GFP-only retrovirus (OP9-EV). When hES cells were plated onto OP9-EV feeder layers in serum-free medium containing bFGF but no LIF or BMP4, the majority of cells maintained an ES-like morphology after 1 wk. In contrast, when cells were plated onto OP9-D11 feeders in the same medium, the edges of the colonies, where they contact the GFP+ OP9-D11 feeder cells, underwent a morphological change within the first week (FIG. 10). They became compact and elongated with barely distinguishable nuclei (FIG. 10A, region between dotted lines). Antibody staining confirmed that these cells were negative for the ES markers TRA1/60 and TRA 1/81 (unpublished data) and positive for Sox1, Nestin, and Pax6 (FIG. 10B, 10C, and unpublished data). In human ES cells, Pax6 is the earliest known marker of neural differentiation, appearing several days before Sox1 begins to be expressed. We carried out quantification of this marker using image analysis software. This confirmed a significant increase in both the number of Pax6+ colonies (colonies containing more than ten Pax6+ cells) and in the area that is Pax6+ within each of these colonies in OP9-D11-supported cultures in comparison with OP9-EV colonies (FIG. 10D-10G, 10J, and 10K, p<0.01). The positive effect of OP9-D11 feeders on neural differentiation appeared to be specifically due to activation of Notch signalling, because it could be blocked by adding the gamma secretase inhibitor (FIG. 10H-10K, p<0.01). Exposure to neither Deltal nor the γ-secretase inhibitor had any discernible effect on cell number or viability.

We went on to test whether endogenous Notch signalling is required for neural differentiation in hES cells. For these experiments, we made use of a monolayer neural differentiation protocol adapted from that for mouse ES cells. Briefly, we removed the hES from feeders and from exogenous LIF and BMP4 and plated them onto Matrigel in FGF-only serum-free medium. Under these conditions, typically around 60% of the culture area loses expression of the ES cell marker TRA1/81, adopts a neural morphology, and becomes Sox1+ Pax6+ after 1 wk (FIG. 10L, 10M, 10P, 10R, and 10T). In contrast, in the presence of the γ-secretase inhibitor, there is a significant reduction in emergence of Pax6+ regions within the culture (FIGS. 10M, 10O, 10Q, 10S, and 10T, p<0.5), with a corresponding increased persistence of undifferentiated TRA1/81+ES cells (FIG. 10O). The γ-secretase inhibitor had no obvious effect on cell viability or cell number, and the majority of treated cells retained a healthy hES-like morphology (FIG. 10M).

These data show that Notch promotes neural differentiation in human ES cells.

### Example 8

NotchIC Suppresses Nonneural Differentiation

We carried out quantitative RT-PCR to measure the expression of endoderm and mesoderm markers on the sixth day of monolayer differentiation. Several nonneural markers were readily detected in parental cell samples, in marked contrast to the barely detectable expression levels in R26NotchIC cell. These observations indicate that not only does Notch promote neural lineage entry but it also simultaneously suppresses nonneural commitment.

R26-NotchIC ES also showed a marked reduction in mesoderm differentiation when tested under an inductive differentiation protocol based on monolayer culture on collagen IV in the presence of batch-tested serum.

### REFERENCES

Bain, G., Ray, W. J., Yao, M., and Gottlieb, D. I. (1996). Retinoic acid promotes neural and represses mesodermal gene expression in mouse embryonic stem cells in culture. Biochem Biophys Res Commun 223, 691-4.
Bibel, M., Richter, J., Schrenk, K., Tucker, K. L., Staiger, V., Korte, M., Goetz, M., and Barde, Y. A. (2004). Differentiation of mouse embryonic stem cells into a defined neuronal lineage. Nat Neurosci 7, 1003-9.
Gaiano, N., Nye, J. S., and Fisbell, G. (2000). Radial glial identity is promoted by Notch1 signalling in the murine forebrain. Neuron 26, 395-404.
Hitoshi, S., Alexson, T., Tropepe, V., Donoviel, D., Elia, A. J., Nye, J. S., Conlon, R. A., Mak, T. W., Bernstein, A., and van der Kooy, D. (2002). Notch pathway molecules are essential for the maintenance, but not the generation, of mammalian neural stem cells. Genes Dev 16, 846-58.
Kopan, R., Nye, J. S., and Weintraub, H. (1994). The intracellular domain of mouse Notch: a constitutively activated repressor of myogenesis directed at the basic helix-loop-helix region of MyoD. Development 120, 2385-96.
Lai, E. C. (2004). Notch signalling: control of cell communication and cell fate. Development 131, 965-73.
Li, Y., and Baker, N. E. (2001). Proneural enhancement by Notch overcomes Suppressor-of-Hairless repressor function in the developing Drosophila eye. Curr Biol 11, 330-8.
Morrison, S. J., Perez, S. E., Qiao, Z., Verdi, J. M., Hicks, C., Weinmaster, G., and Anderson, D. J. (2000). Transient Notch activation initiates an irreversible switch from neurogenesis to gliogenesis by neural crest stem cells. Cell 101, 499-510.
Nakamura, Y., Sakakibara, S., Miyata, T., Ogawa, M., Shimazaki, T., Weiss, S., Kageyama, R., and Okano, H. (2000). The bHLH gene hesI as a repressor of the neuronal commitment of CNS stem cells. J Neurosci 20, 283-93.
Ohtsuka, T., Sakamoto, M., Guillemot, F., and Kageyama, R. (2001). Roles of the basic helix-loop-helix genes Hes1 and Hes5 in expansion of neural stem cells of the developing brain. J Biol Chem 276, 30467-74.
Schroeder, T., Fraser, S. T., Ogawa, M., Nishikawa, S., Oka, C., Bornkamm, G. W., Honjo, T., and Just, U. (2003). Recombination signal sequence-binding protein Jkappa alters mesodermal cell fate decisions by suppressing cardiomyogenesis. Proc Natl Acad Sci USA 100, 4018-23.
Smith, A. G. (1991). Culture and differentiation of embryonic stem cells. J. Tiss. Cult. Meth 13, 89-94.
Tanigaki, K., Nogaki, F., Takahashi, J., Tashiro, K., Kurooka, H., and Honjo, T. (2001). Notch1 and Notch3 instructively restrict bFGF-responsive multipotent neural progenitor cells to an astroglial fate. Neuron 29, 45-55.
Ying, Q. L., Nichols, J., Chambers, I., and Smith, A. (2003a). BMP induction of Id proteins suppresses differentiation and sustains embryonic stem cell self-renewal in collaboration with STAT3. Cell 115, 281-92.
Ying, Q. L., and Smith, A. G. (2003). Defined conditions for neural commitment and differentiation. Methods Enzymol 365, 327-41.
Ying, Q. L., Stavridis, M., Griffiths, D., Li, M., and Smith, A. (2003b). Conversion of embryonic stem cells into neuroectodermal precursors in adherent monoculture. Nat Biotechnol 21, 183-6.

The invention thus provides neural directed differentiation of pluripotent cell cultures, and methods and compositions therefor.

1-6. (canceled)

7. A method of obtaining a culture enriched in neural cells, comprising:
(a) providing a culture comprising multipotent or pluripotent cells which have the potential to form progeny committed to either a neural or a non-neural fate; and
(b) activating Notch signalling in the cells.

8. A method according to claim 7, wherein the activating comprises expressing in the cells a Notch receptor or an activated form of a Notch receptor.

9. (canceled)

10. A method according to claim 7, wherein the activating comprises adding to culture medium a notch ligand.

11. (canceled)

12. A method according to claim 7, wherein the activating comprises expressing in the cells a downstream effector of Notch signalling.

13. A method according to claim 7, wherein the activating comprises adding to culture medium a composition comprising a downstream effector of Notch signalling and a transduction domain.

14. (canceled)

15. A method according to claim 7, wherein the activating comprises adding to culture medium a composition an activated form of a Notch receptor and a transduction domain.

16-18. (canceled)

16. A method according to claim 7 further comprising purifying the neural cells.

20-21. (canceled)

22. A method according to claim 7 comprising culturing the cells in medium non-permissive for the multipotent or the pluripotent cells.

23-27. (canceled)

28. A method of increasing the density of neural progenitor cells in culture, comprising activating Notch signalling in the cells.

29. A method according to claim 28, wherein the cells are human cells.

30. A composition comprising an activated form of a Notch receptor and a transduction domain.

31. A composition according to claim 30, wherein the activated form of a Notch receptor comprises an intracellular domain of a Notch receptor.

32. A composition comprising a downstream effector of Notch signalling and a transduction domain.

33. A composition according to claim 32, wherein the effector is a Hes transcription factor or a Hey transcription factor.

34-36. (canceled)

37. A fusion protein of an activated form of a Notch receptor and a transduction domain.

38. A fusion protein of a downstream effector of Notch signalling and a transduction domain.

39. A nucleotide sequence encoding the fusion protein of claim 37.

40. A vector comprising the nucleotide sequence of claim 39.

41. (canceled)

42. A medium for culture of neural cells, comprising a composition according to claim 30

43. A medium according to claim 42, wherein the medium is non-permissive for multipotent cells.

44. A medium according to claim 42, wherein the medium is non-permissive for pluripotent cells.

45. A pluripotent cell in which Notch signalling has been activated.

46. An ES cell according to claim 45.

47. (canceled)

48. (canceled)

49. A pluripotent cell engineered to express a peptide comprising a Notch intracellular domain.

50. An ES cell according to claim 49.

51. (canceled)

52. (canceled)
